(19) United States
(12) Patent Application Publication
(10) Pub. No.: US 2021/0048410 A1
(43) Pub. Date: Feb. 18, 2021

(54) Tissue Projection Electrophoretic Separation of Protein

(71) Applicant: The Regents Of The University Of California, Oakland, CA (US)

(72) Inventors: Amy E. Herr, Oakland, CA (US); Samantha Marie Grist, Berkeley, CA (US); Chi-Chih Kang, Berkeley, CA (US); Burcu Gumuscu Sefunc, Enschede (NL); Julea Vlassakis, Berkeley, CA (US)

(21) Appl. No.: 16/977,175

(22) PCT Filed: Apr. 29, 2019
(86) PCT No.: PCT/US19/29662

§ 371 Date: Sep. 1, 2020

Related U.S. Application Data
(60) Provisional application No. 62/664,835, filed on Apr. 30, 2018.

(51) Int. Cl. G01N 27/453; G01N 33/48; B01L 3/00

(52) U.S. Cl. CPC G01N 27/453; B01L 2300/0829; B01L 3/502715; G01N 33/48

(57) ABSTRACT

Electrophoretic separation methods and devices for detecting a distribution of analytes in a tissue sample are provided. The methods and devices find use in a variety of different electrophoretic separation applications, such as tissue projection electrophoretic separation of proteins from a tissue sample, where analytes in a tissue sample can be detected to produce a map of the distribution of the analytes in the tissue sample.

### CROSS-REFERENCE TO RELATED APPLICATIONS

This application claims the benefit of U.S. Provisional Application No. 62/664,835, filed on Apr. 30, 2018, the disclosure of which is incorporated herein by reference in its entirety.

### REFERENCE TO GOVERNMENT SUPPORT

This invention was made with government support under Grant Number CA225296 awarded by the National Institutes of Health. The government has certain rights in the invention.

### INTRODUCTION

A variety of analytical techniques may be used to separate and detect specific analytes in a given sample. A range of related immunoblotting methods have enabled the identification and semi-quantitative characterization of e.g., DNA (Southern blot), RNA (northern blot), proteins (Western blot), and protein-protein interactions (far-western blot); by coupling biomolecule separations and assays. For example, Western blotting can be used to detect proteins in a sample by using gel electrophoresis to separate the proteins in the sample followed by probing with antibodies specific for the target protein. In a typical Western blot, gel electrophoresis is used to separate native proteins by 3-D structure or denatured proteins by the length of the polypeptide. The proteins are then transferred to a membrane (typically nitrocellulose or PVDF), where they are probed (detected) using antibodies specific to the target protein.

There are a wide number of alternative splicing events, post-translational modifications, and co-translational modifications (e.g., phosphorylation, glycosylation, and protein cleavage) that give rise to ‘proteoforms’ with distinct function and subsequent cell behavior but that cannot be resolved with conventional methods such as immunohistochemistry (IHC). Protein complexes also cannot be resolved with conventional IHC but it is their formation that drives key cell behaviors such as transcription and translation. Despite their importance in biological processes, these kinds of proteoforms and complexes are difficult and, in many cases, impossible to measure in tissues by conventional IHC and pathology. Analytical variability (lack of isoform- or complex-specific antibody probes), biological variability (small cell subpopulations diluted in bulk analysis), and lack of multiplexing (measurement of multiple proteins from the same tissues) can all render proteoforms and protein complexes undetectable by current technologies.

### SUMMARY

Electrophoretic separation methods and devices for detecting a distribution of analytes in a tissue sample are provided. The methods and devices find use in a variety of different electrophoretic separation applications, such as tissue projection electrophoretic separation of proteins from a tissue sample.

Aspects of embodiments of the present disclosure include a method of detecting a distribution of analytes in a tissue sample. The method includes: (a) applying an electric field to a tissue sample on a polymeric separation medium in a manner sufficient to move one or more analytes from the tissue sample into the polymeric separation medium to separate the analytes in the polymeric separation medium; (b) exposing the polymeric separation medium to an applied stimulus to immobilize the separated analytes in the polymeric separation medium; and (c) detecting the analytes to produce a map of the distribution of the analytes in the tissue sample.

In some embodiments, the map is a three-dimensional distribution of the analytes in the tissue sample.

In some embodiments, the polymeric separation medium includes a buffer sufficient to differentially lyse different cell types in the tissue sample. In some embodiments, the polymeric separation medium includes a buffer sufficient to differentially lyse a subcellular compartment of one or more cells in the tissue sample. In some embodiments, the polymeric separation medium includes a buffer sufficient to either retain or dissociate protein complexes in the tissue sample. In some embodiments, the polymeric separation medium includes a buffer sufficient for separation of cellular proteins from matrix proteins in the tissue sample.

In some embodiments, the detecting includes contacting the separated analytes with an analyte detection reagent. In some embodiments, the detecting also includes imaging the polymeric separation medium to produce an image of the separated analytes in the polymeric separation medium.

In some embodiments, the method includes assembling a device for performing the method. In some embodiments, the assembling includes: (1) disposing the tissue sample on a first electrode; (2) positioning the tissue sample in fluid communication with a first surface of the polymeric separation medium; and (3) disposing a second electrode on an opposing second surface of the polymeric separation medium.

In some embodiments, the tissue sample is disposed on a surface of a support medium. In some embodiments, the tissue sample is contained in a support medium.

Aspects of the present disclosure include a device for detecting a distribution of analytes in a tissue sample, where the device includes: a tissue sample that includes one or more analytes; and a polymeric separation medium that includes a buffer and functional groups that covalently bond the analytes in the tissue sample to the polymeric separation medium upon application of an applied stimulus.

In some embodiments, one surface of the tissue sample is in contact with a surface of the polymeric separation medium and an opposing surface of the tissue sample is in contact with a support medium.

In some embodiments, the tissue sample is contained in a support medium, and a surface of the support medium is in contact with a surface of the polymeric separation medium.

In some embodiments, the polymeric separation medium includes an array of microwells in a surface of the polymeric separation medium facing the tissue sample.

In some embodiments, the functional groups comprise light-activated benzophenone functional groups.

In some embodiments, the polymeric separation medium includes one or more separated analytes from the tissue sample, and the separated analytes in the polymeric separation medium are correlated to a distribution of the analytes in the tissue sample.

In some embodiments, the device also includes a chamber containing the tissue sample and the polymeric separation medium.

Aspects of the present disclosure include a kit for performing the method according to the present disclosure, where the kit includes a device having the polymeric separation medium as described herein, and a packaging containing the device.

In some embodiments, the kit also includes one or more buffers.

### BRIEF DESCRIPTION OF THE FIGURES

FIG. 1, panels A to D, show a schematic flow diagram illustrating a partial glass silanization process for released gel fabrication, according to embodiments of the present disclosure.

FIG. 2, panels A to F, show a schematic flow diagram illustrating a fabrication process for released, micropatterned gels for Z-directional electrophoresis in a tissue projection device, according to embodiments of the present disclosure. Thick gels were chemically polymerized by molding to a microfabricated SU-8 photoresist on a glass mold, and demolded using a partially-silanized glass slide. The partially-silanized glass slide facilitated gel release from the glass after demolding.

FIG. 3 shows a schematic illustration of a tissue projection electrophoresis device, according to embodiments of the present disclosure.

FIG. 4 shows a schematic illustration of an electrophoresis device for analysis of purified proteins, according to embodiments of the present disclosure.

FIG. 5, panels A to F, show a schematic flow diagram illustrating the steps of a tissue projection electrophoresis separation method, according to embodiments of the present disclosure.

### DETAILED DESCRIPTION

Electrophoretic separation methods and devices for detecting a distribution of analytes in a tissue sample are provided. The methods and devices find use in a variety of different electrophoretic separation applications, such as tissue projection electrophoretic separation of proteins from a tissue sample.

In addition to the methods described herein, electrophoretic separation devices and kits using the same are provided. Below, the subject electrophoretic separation devices are described first in greater detail. Methods of detecting a distribution of analytes in a tissue sample are also disclosed in which the subject devices find use. In addition, kits that include the subject devices are also described.

### Devices

Embodiments of the present disclosure include devices, such as separation devices useful for detecting a distribution of analytes in a tissue sample. As such, in certain embodiments, the separation devices are configured to separate analytes from a tissue sample. For example, the separation devices may be configured to separate analytes in a tissue sample based on one or more physical and/or chemical properties of the analytes. In some instances, the analytes may include detectable differences in their molecular weight, size, charge (e.g., mass to charge ratio), isoelectric point, affinity interactions, and the like. Separation devices of the present disclosure may be configured to distinguish different analytes from each other based on one or more of their molecular weight, size, charge (e.g., mass to charge ratio), isoelectric point, affinity interactions, and the like. In some instances, a “tissue sample” is a collection of a plurality of cells obtained from a subject to be analyzed. A tissue sample may be a sample of tissue from a subject where the position and arrangement of the cells in the sample are substantially the same as the position and arrangement of the cells before the sample was removed from the subject. In some instances, a tissue sample is more than a single cell. As such, in certain embodiments, devices according to the present disclosure are configured for analysis of a collection of associated cells, rather than for single-cell analysis.

A tissue sample can be obtained from a subject by excision of the tissue sample from the subject. For instance, a tissue sample can be obtained from the subject by removing the tissue sample from the subject, such as by cutting the tissue sample out of the subject. In some instances, a biopsy is performed, for example where the normal tissue structure is preserved, which provides for examination of both individual cells and their organization (e.g., 2-dimensional and/or 3-dimensional organization) in the tissue structure. The tissue sample can be a solid tissue sample or a soft tissue sample. For example, in some cases, the tissue sample analyzed using a device of the present disclosure is a tissue slice obtained from a subject.

Devices of the present disclosure are also configured for detecting a distribution of analytes in a tissue sample. For instance, analytes can be separated from a tissue sample while at the same time substantially maintaining their relative positions with respect to each other as they would be found in the tissue sample before the tissue sample was subjected to analysis. The term “tissue projection” is meant to describe the separation of analytes from a tissue sample, such that when analytes are separated from the tissue sample in a direction orthogonal to a surface of the tissue sample, the analytes substantially maintain their relative positions with respect to each other when viewed from above (i.e., in a plan view). In some instances, analytes of the tissue sample are separated in the Z direction relative to the X-Y plane of the tissue sample. As such, devices of the present disclosure are configured for detecting the distribution of analytes in a tissue sample, where the distribution of the analytes separated from the tissue sample is substantially the same as the distribution of the analytes in the tissue sample before analysis. In some instances, devices of the present disclosure facilitate analysis of a tissue sample by providing for the detection and production of a map, such as a two-dimensional (2D) or three-dimensional (3D) map, of the relative distribution of analytes in the tissue sample.

As described above, the separation devices are configured to separate analytes from a tissue sample. In some instances, the device includes a polymeric separation medium in which the analytes are separated from each other. Aspects of the polymeric separation medium are described in more detail below.

### Polymeric Separation Medium

The polymeric separation medium may be configured to separate constituents (e.g., analytes) in a tissue sample from each other. In some cases, the separation medium is configured to separate constituents in a tissue sample based on the physical properties of the constituents. For example, the separation medium may be configured to separate the constituents in the sample based on the molecular mass, size, charge (e.g., charge to mass ratio), isoelectric point, affinity interactions, etc. of the constituents.

In certain instances, the separation medium is configured to separate the constituents in the tissue sample based on the size and charge of the constituents. The separation medium may be configured to separate the constituents in the tissue sample into distinct detectable bands or zones of constituents. By “band” or “zone” is meant a distinct detectable region where the concentration of a constituent is significantly higher than the surrounding regions. Each band or zone of constituent may include a single constituent or several constituents, where each constituent in a single band or zone of constituents has substantially similar physical properties, as described above.

In certain embodiments, the separation medium is configured to separate the constituents in a tissue sample as the analytes traverse the separation medium. In some cases, the separation medium is configured to separate the constituents in the tissue sample as the analytes flow through the separation medium. Aspects of the separation medium include that the separation medium has a directional separation axis, as described in more detail below. In some instances, the directional separation axis is oriented in the direction the analytes travel as the analytes traverse the separation medium. For example, the separation axis may be oriented in a direction orthogonal to a surface of the tissue sample. In some instances, a tissue sample may be substantially planar (e.g., a tissue slice), and the separation axis may be aligned in a direction substantially perpendicular to the plane of the tissue sample (e.g., substantially orthogonal to the tissue slice). For instance, as described above, the tissue sample may be substantially planar, and thus can be defined as an x-y plane. In these instances, the separation axis, which is substantially perpendicular to the plane of the tissue sample, can be defined as the z-axis.

Polymeric Separation Medium with a Planar Array of Microwells

In certain embodiments, the polymeric separation medium includes an array of microwells, such as a planar array of microwells. The array of microwells may be formed in a surface of the polymeric separation medium, such as in the surface of the polymeric separation medium facing or in contact with the tissue sample. In some instances, the array of microwells facilitates an improvement in x-y resolution by reducing diffusion of analytes in the sample in the x-y tissue plane.

In certain embodiments, the directional separation axis is aligned with the thickness direction of the separation medium. For instance, the directional separation axis may be substantially parallel to the z-axis of the separation medium; i.e., substantially perpendicular to the x-y plane of the tissue sample. In some embodiments, the separation medium is square or rectangular in shape (when viewed from above in a plan view) and the directional axis of the separation medium may be aligned with the height or thickness direction of the separation medium. Other shapes (when viewed from above in a plan view) of the separation medium are also contemplated, such as circular, elliptical, triangular, or irregular shapes. In some embodiments, the analytes traverse the separation medium in the height or thickness direction (i.e., along the z-axis). In some cases, where the sample traverses the height or thickness of the separation medium, the height or thickness of the separation medium is greater than the width or length of the separation medium, such as 2 times, 3 times, 4 times, 5 times, 10 times, 25 times, 50 times, 75 times, 100 times, 125 times, 150 times, 175 times, or 200 times or more than the width or length of the separation medium. In some instances, a longer separation axis may facilitate an increase in resolution between bands or zones of different analytes in the tissue sample. In other cases, the height or thickness of the separation medium is less than the width or length of the separation medium, such as 2 times, 3 times, 4 times, 5 times, 10 times, 25 times, 50 times, 75 times, 100 times, 125 times, 150 times, 175 times, or 200 times or less than the width or length of the separation medium.

In certain embodiments, the separation medium includes a plurality of microwells in the separation medium. In some instances, the separation medium includes a substantially planar array of microwells in the separation medium. An “array of microwells” includes any two-dimensional or substantially two-dimensional arrangement of microwells. For example, a planar array of microwells may be arranged into rows and columns of microwells. The microwells in the planar array of microwells may be individually addressable. A microwell is “addressable” when the array includes multiple microwells positioned at particular predetermined locations (e.g., “addresses”) in the array. Microwells may be separated by intervening spaces. A planar array of microwells may include one or more, including two or more, four or more, eight or more, 10 or more, 25 or more, 50 or more, 100 or more, 200 or more, 300 or more, 400 or more, 500 or more, 750 or more, 1000 or more, 1500 or more, 2000 or more, 2500 or more, 3000 or more, 3500 or more, 4000 or more, 4500 or more, 5000 or more, 5500 or more, 6000 or more, 6500 or more, 7000 or more, 7500 or more, 8000 or more, 8500 or more, 9000 or more, 9500 or more, 10,000 or more, or 25,000 or more, or 50,000 or more, or 75,000 or more, or 100,000 or more microwells in a polymeric separation medium. In some cases, a planar array of microwells may include 5000 or more microwells. A polymeric separation medium may include one or more arrays of microwells, for example, 1 or more, 2 or more, 3 or more, 4 or more, 5 or more, 6 or more, 7 or more, 8 or more, 9 or more, 10 or more, 12 or more, 14 or more, 16 or more, 18 or more, 20 or more, 25 or more, 30 or more, 35 or more, 40 or more, 45 or more, 50 or more, 75 or more, or 100 or more arrays of microwells. In some cases, the polymeric separation medium includes 10 or more arrays of microwells. Depending upon the use, any or all of the microwells may be the same or different from one another and each may be configured to contain distinct analytes or sample constituents. Aspects of individual microwells are described in more detail below, but may be applied to any or all of the microwells in the array of microwells.

In certain embodiments, the polymeric separation medium includes a planar array of microwells as described above. The planar array of microwells may be arranged such that each microwell has an open end provided on a surface of the separation medium (e.g., on a surface of the separation medium which faces the tissue sample, such as on a bottom surface of the separation medium). In these embodiments, the interior volume of each microwell may extend from the open end of the microwell on the surface of the polymeric separation medium into the polymeric separation medium. In certain embodiments, the open end of the microwell (and thus the interior volume of the microwell) is in fluid communication with a fluid provided on or around the surface of the separation medium (e.g., buffer, sample, etc.). In some instances, the closed end of the microwell is formed by the material of the polymeric separation medium, e.g., in embodiments where the interior volume of the microwell does not extend all the way through the separation medium, such as where the depth of the microwell is less than the thickness of the polymeric separation medium.

In certain embodiments, the microwell is configured such that an axis of the microwell from the closed end to the open end of the microwell is substantially perpendicular to the surface of the separation medium (e.g., substantially perpendicular to the surface of the separation medium having the open ends of the microwells). In certain embodiments, the walls (e.g., the side walls) of the microwell are formed by the material of the polymeric separation medium, such as where the interior volume of the microwell extends into the polymeric separation medium and is surrounded by the polymeric separation medium.

In certain embodiments, the microwell has an interior volume with a defined shape. For example, the interior volume of the microwell may have a shape of a cylinder, a cube, a rectangular cuboid, a frustum (e.g., a square frustum, a rectangular frustum, a conical frustum, etc.), and the like. The microwells in an array of microwells can all have the same shape, or combinations of different shaped microwells can be present in an array of microwells.

In certain embodiments, the open end of the microwell has dimensions greater than the closed end of the microwell. For instance, the open end of the microwell may have dimensions (e.g., width and/or length, or diameter, depending on the shape of the microwell) that are 1.1 times greater than the dimensions of the closed end of the microwell, such as 1.2 times, or 1.3 times, or 1.4 times, or 1.5 times, or 1.6 times, or 1.7 times, or 1.8 times, or 1.9 times, or 2 times the dimensions of the closed end of the microwell.

A “microwell” is a well that has dimensions in the micrometer scale. While the dimensions may vary, in some instances, the open end of the microwell has a width of 100 μm or less, such as 90 μm or less, or 80 μm or less, or 70 μm or less, or 60 μm or less, or 50 μm or less, or 40 μm or less, or 30 μm or less, or 20 μm or less, or 10 μm or less. For example, the open end of the microwell may have a width ranging from 10 μm to 100 μm, such as 10 μm to 90 μm, or 10 μm to 80 μm, or 10 μm to 70 μm, or 10 μm to 60 μm, or 10 μm to 50 μm, or 10 μm to 40 μm, or 10 μm to 30 μm.

In some cases, the closed end of the microwell has a width of 100 μm or less, such as 90 μm or less, or 80 μm or less, or 70 μm or less, or 60 μm or less, or 50 μm or less, or 40 μm or less, or 30 μm or less, or 20 μm or less, or 10 μm or less. For example, the closed end of the microwell may have a width ranging from 10 μm to 100 μm, such as 10 μm to 90 μm, or 10 μm to 80 μm, or 10 μm to 70 μm, or 10 μm to 60 μm, or 10 μm to 50 μm, or 10 μm to 40 μm, or 10 μm to 30 μm, or 10 μm to 20 μm.

In certain embodiments, the microwell has a depth (e.g., the distance from the open end to the closed end of the microwell) of 100 μm or less, such as 90 μm or less, or 80 μm or less, or 70 μm or less, or 60 μm or less, or 50 μm or less, or 40 μm or less, or 30 μm or less, or 20 μm or less, or 10 μm or less. For example, the microwell may have a depth ranging from 10 μm to 100 μm, such as 10 μm to 90 μm, or 10 μm to 80 μm, or 10 μm to 70 μm, or 10 μm to 60 μm, or 20 μm to 60 μm, or 30 μm to 60 μm, or 40 μm to 60 μm.

In certain embodiments, the size and shape of the microwells is such that each microwell can accommodate a single cell. The microwells in the polymeric separation medium may be substantially uniform. For example, the shape and size of the microwells in the separation medium may be substantially uniform. In other embodiments, the microwells may be different, such as having a different shape, a different size, combinations thereof, and the like. A separation medium that includes different microwells may facilitate the analysis of different sample constituents at the same time. For instance, microwells that have different shapes and/or sizes may preferentially capture different shaped or sized sample components (e.g., different shaped or sized cells in the sample).

In certain embodiments, the separation medium includes a polymer, such as a polymeric gel. The polymeric gel may be a gel suitable for gel electrophoresis. The polymeric gel may include, but is not limited to, a polyacrylamide gel (e.g., methacrylamide gel), an agarose gel, and the like. The resolution of the separation medium may depend on various factors, such as, but not limited to, pore size, total polymer content (e.g., total acrylamide content), concentration of cross-linker, applied electric field, assay time, and the like. For instance, the resolution of the separation medium may depend on the pore size of the separation medium. In some cases, the pore size depends on the total polymer content of the separation medium and/or the concentration of crosslinker in the separation medium. In certain instances, the separation medium is configured to resolve analytes with molecular mass differences of 50,000 Da or less, or 25,000 Da or less, or 10,000 Da or less, such as 7,000 Da or less, including 5,000 Da or less, or 2,000 Da or less, or 1,000 Da or less, for example 500 Da or less, or 100 Da or less. In some cases, the separation medium may include a polyacrylamide gel that has a total acrylamide content, T (T=total concentration of acrylamide and bisacrylamide monomer, % w/v), ranging from 1% to 20%, such as from 3% to 15%, including from 5% to 15%, or from 5% to 10%, or from 10% to 15%, or from 15% to 20%. In certain embodiments, the separation medium includes a polyacrylamide gel that has a crosslinker content, C (% w/v), ranging from 1% to 10%, such as from 2% to 7%, including from 2% to 5%.

In certain embodiments, the separation medium is configured to be formed from precursor moieties. For example, the separation medium may be a gel (e.g., a polyacrylamide gel) formed form gel precursors (e.g., polyacrylamide gel precursors, such as polyacrylamide gel monomers). The precursor moieties may be configured to react to form the separation medium. For instance, the gel precursors may be configured to react with each other to form the polyacrylamide gel separation medium. The reaction between the gel precursors may be activated by any suitable protocol, such as, but not limited to, chemical activation, light activation, etc. In some embodiments, the gel precursors are configured to be activated chemically, for example by contacting the gel precursors with an activation agent, such as, but not limited to, a peroxide. In some embodiments, the gel precursors are configured to be activated by light (i.e., photo-activated), for instance by contacting the gel precursors with light. The light may be of any wavelength suitable for activating the formation of the separation medium, and in some instances may have a wavelength associated with blue light in the visible spectrum. For example, the light used to activate formation of the separation medium may have a wavelength ranging from 400 nm to 500 nm, such as from 410 nm to 490 nm, including from 420 nm to 480 nm, or from 430 nm to 480 nm, or from 440 nm to 480 nm, or from 450 nm to 480 nm, or from 460 nm to 480 nm, or from 465 nm to 475 nm. In certain cases, the light used to activate formation of the separation medium has a wavelength ranging from 465 to 475 nm. In some instances, the light used to activate formation of the separation medium has a wavelength of 470 nm.

The dimensions of the separation medium may be varied depending on the assay being performed and/or the size of the tissue sample to be analyzed. In some instances, the dimensions of the separation medium are such that the separation medium has a length and a width substantially the same as the tissue sample being analyzed. In some instances, the dimensions of the separation medium are such that the separation medium has a length and a width greater than the length and the width of the tissue sample being analyzed. For example, the edges of the separation medium may extend beyond the edges of the tissue sample such that the entire surface of the tissue sample is covered by the separation medium. In some instances, the separation medium has dimensions in the range of 10 mm×10 mm to 200 mm×200 mm, including dimensions of 100 mm×100 mm or less, such as 50 mm×50 mm or less, for instance 25 mm×25 mm or less, or 10 mm×10 mm or less, or 5 mm×5 mm or less, for instance, 1 mm×1 mm or less. In some cases, the separation medium has a thickness ranging from 1 μm to 100 mm, such as from 10 μm to 100 mm, or from 100 μm to 75 mm, or from 500 μm to 50 mm, or from 500 μm to 10 mm.

In certain embodiments, the separation medium includes a buffer. The buffer may be any convenient buffer used for gel electrophoresis. In certain embodiments, the buffer is a Tris buffer. In certain embodiments, the separation medium includes a buffer, such as a Tris-glycine buffer. For example, the buffer may include a mixture of Tris and glycine.

In certain embodiments, the buffer is sufficient to perform both lysis of cells or a portion thereof (e.g., differential lysis of a sub-cellular compartment) and electrophoresis of the cellular components released by lysis of the cell or portion thereof (e.g., differential lysis of a sub-cellular compartment).

In some cases, the buffer is a buffer sufficient to differentially lyse a sub-cellular compartment of a cell to produce a set of cellular components. For instance, the buffer may be configured to lyse a first sub-cellular compartment, such as the cell membrane, without causing significant lysis of other sub-cellular compartments, such as the nuclear membrane. In some cases, the buffer is configured to selectively lyse the cell membrane such that cytosol is released from the cell without causing significant lysis of other sub-cellular compartments, such as the nuclear membrane. In certain embodiments, a second buffer may be used, where the second buffer may be configured to lyse a different sub-cellular compartment, such as the nuclear membrane. In certain embodiments, the second buffer does not cause significant lysis of other sub-cellular compartments, such as mitochondria, plastids, or other organelles. In some cases, the second buffer is configured to selectively lyse the nuclear membrane such that the contents of the cell nucleus are released from the nucleus without causing significant lysis of other sub-cellular compartments, such as mitochondria, plastids, or other organelles. Different buffers may be used sequentially in different steps of the methods described herein to achieve differential lysis of sub-cellular compartments of the cell, such that the contents of different sub-cellular compartments of a cell may be separately analyzed in series.

In certain embodiments, the buffer is sufficient to differentially lyse different cell types in a tissue sample. For instance, the buffer may be configured to lyse a first cell type without causing significant lysis of other cell types in the tissue sample. In some cases, the buffer is configured to selectively lyse a first cell type such that cellular components are released from the first cell type without causing significant lysis of other cell types in the tissue sample. In certain embodiments, a second buffer may be configured to lyse a different (second) cell type. In certain embodiments, the second buffer does not cause significant lysis of other cell types, including the first cell type. In some cases, the second buffer is configured to selectively lyse the second cell type such that the contents of the second cell type are released without causing significant lysis of other cell types in the tissue sample. Different buffers may be used sequentially in different steps of the methods described herein to achieve differential lysis of different cell types, such that the contents of different cell types may be analyzed separately in series.

In certain embodiments, the buffer is sufficient to either retain or dissociate protein complexes in a tissue sample. For instance, the buffer may be configured to retain protein complexes without causing significant dissociation of protein complexes in the sample. In other cases, the buffer is configured to dissociate protein complexes in the sample. A “protein complex” is an association of two of more proteins into a three-dimensional structure. The proteins in a protein complex can be stably associated with each other. By “stably associated” is meant that a protein in a protein complex is non-covalently bound to or otherwise associated with another protein under standard conditions. Non-covalent interactions may include, but are not limited to, ionic bonds, hydrophobic interactions, hydrogen bonds, van der Waals forces (e.g., London dispersion forces), dipole-dipole interactions, and the like.

In certain embodiments, the buffer is sufficient for separation of cellular proteins from matrix proteins in a tissue sample. For instance, the buffer may be configured to separate cellular proteins from the tissue sample. In other cases, the buffer may be configured to separate matrix proteins from the tissue sample. Different buffers may be used sequentially in different steps of the methods described herein to achieve differential analysis of cellular proteins and matrix proteins, such that cellular proteins and matrix proteins may be analyzed in series. As used herein, “cellular proteins” are proteins that are contained within a cell or expressed on a surface of the cell. As used herein “matrix proteins” are proteins in a tissue sample that are in the extracellular matrix surrounding cells in the tissue sample.

In some embodiments, the buffer is used at room temperature. In some embodiments, the buffer is heated to a temperature above room temperature. For instance, the buffer may be heated to a temperature of 25° C. or more, or 30° C. or more, or 35° C. or more, or 40° C. or more, or 45° C. or more, or 50° C. or more, or 55° C. or more, or 60° C. or more, or 65° C. or more, or 70° C. or more, or 75° C. or more. In some cases, the buffer is heated to 50° C.

In some cases, the buffer includes a detergent. In certain instances, the detergent is configured to provide analytes in the sample with substantially similar charge-to-mass ratios. Analytes with substantially similar charge-to-mass ratios may facilitate the separation of the analytes into one or more bands or zones in the separation medium based on the molecular masses of the analytes in the sample. In certain cases, the detergent is an anionic detergent configured to provide analytes in the sample with a charge, such as a negative charge. For example, the detergent may be an anionic detergent, such as, but not limited to, sodium dodecyl sulfate (SDS). In some instances, the detergent may be digitonin. In some instances, the detergent may be sodium deoxycholate. Combinations of detergents may also be included in the buffer.

In certain embodiments, the buffer is configured to selectively lyse the cell membrane such that cytosol is released from the cell without causing significant lysis of other sub-cellular compartments, such as the nuclear membrane; e.g., the buffer is a cytosol lysis buffer. Examples of cytosol lysis buffers include, but are not limited to, Triton X-100, Tris-glycine, and the like, and combinations thereof. In some instances, the cytosol lysis buffer may include a detergent, such as, but not limited to digitonin. For example, a cytosol lysis buffer may include Triton X-100, Tris-glycine and digitonin.

In certain embodiments, the buffer is configured to selectively lyse the nuclear membrane such that the contents of the cell nucleus are released from the nucleus without causing significant lysis of other sub-cellular compartments, such as mitochondria, plastids, or other organelles; e.g., the buffer is a nuclear lysis buffer. Examples of nuclear lysis buffers include, but are not limited to, Triton X-100, Tris-glycine, and the like, and combinations thereof. In some instances, the nuclear lysis buffer may include a detergent, such as, but not limited to SDS, sodium deoxycholate, combinations thereof, and the like. For example, a nuclear lysis buffer may include Triton X-100, Tris-glycine, SDS, and sodium deoxycholate.

In certain embodiments, the separation medium is configured to separate the constituents in the sample based on the isoelectric point (pi) of the constituents (e.g., isoelectric focusing, IEF). In some cases, the separation medium includes a polymeric gel as described above. For example, the polymeric gel may include a polyacrylamide gel, an agarose gel, and the like. In certain instances, the polymeric gel includes a pH gradient, which, in some embodiments, is co-polymerized with the polymeric gel. In embodiments where the pH gradient is co-polymerized with the polymeric gel, the pH gradient may be substantially immobilized resulting in a separation medium having an immobilized pH gradient. In certain instances, the pH gradient includes a weak acid or a weak base (e.g., Immobilines), ampholytes, or the like.

In certain embodiments, the separation medium is configured to separate constituents in a sample based on size. For example, in some cases, the separation medium includes a polymeric gel having a pore size gradient. The pore size gradient may decrease along the directional separation axis of the separation medium. For example, the pore size gradient may have a pore size that decreases along the directional separation axis of the separation medium, such that analytes from a sample traversing the separation medium encounter progressively smaller and smaller pore sizes in the separation medium. As constituents in the sample traverse the pore size gradient, the constituents in the sample may be separated based on size. For example, larger constituents in the sample may be retained in the separation medium more readily than smaller constituents, which are able to traverse greater distances through the decreasing pore size gradient.

In some cases, the pore size of the separation medium depends on the total polymer content of the separation medium and/or the concentration of crosslinker in the separation medium. In certain instances, the separation medium pore size sufficient to resolve analytes with molecular mass differences of 50,000 Da or less, or 25,000 Da or less, or 10,000 Da or less, such as 7,000 Da or less, including 5,000 Da or less, or 2,000 Da or less, or 1,000 Da or less, for example 500 Da or less, or 100 Da or less. In some cases, the separation medium may include a polyacrylamide gel that has a pore size that depends on the total acrylamide content, T (T=total concentration of acrylamide and bisacrylamide monomer), where the total acrylamide content, T, ranges from 1% to 20%, such as from 3% to 15%, including from 5% to 10%, or 10% to 15%, or 15% to 20%. In certain embodiments, the separation medium includes a polyacrylamide gel that has a crosslinker content, C (% w/v), ranging from 1% to 10%, such as from 2% to 7%, including from 2% to 5%.

In certain embodiments, the separation medium is configured to covalently bond to the constituents (e.g., analytes) of interest. The covalent bond may be formed upon application of an applied stimulus. For example, the applied stimulus may include electromagnetic radiation, such as light. In some cases, the light is ultraviolet (UV) light. In some instances, the light used to covalently bond the constituents of interest to the separation medium has a wavelength ranging from 10 nm to 400 nm, such as from 50 nm to 400 nm, including from 100 nm to 400 nm, or from 150 nm to 400 nm, or from 200 nm to 400 nm, or from 250 nm to 400 nm, or from 300 nm to 400 nm, or form 325 nm to 375 nm, or from 350 nm to 365 nm. In certain cases, the light has a wavelength ranging from 350 to 365 nm.

In certain embodiments, the light used to covalently bond the constituents of interest to the separation medium has a wavelength different from the light used to activate formation of the separation medium. For example, as described above, the light used to activate formation of the separation medium may have a wavelength of blue light in the visible spectrum. As described above, the light used to covalently bond the constituents of interest to the separation medium may have a wavelength of UV light. As such, in certain embodiments, the separation medium is configured to be formed upon application of a first wavelength of light, and configured to covalently bond the constituents of interest upon application of a second wavelength of light. The first and second wavelengths of light may be blue light and UV light, respectively, as described above.

In some cases, the separation medium includes functional groups that covalently bond to the one or more constituents of interest. For example, the constituents of interest may be an analyte of interest, such as, but not limited to, a protein, a peptide, and the like. The functional groups may include functional groups that are activated upon application of an applied stimulus, such as electromagnetic radiation (e.g., light) as described above. As such, in certain instances, the functional groups are light-activatable functional groups. Upon application of light, the light-activatable functional groups may form a reactive species capable of forming covalent bonds, such as a radical alkyl intermediate. Examples of functional groups that may covalently bond to the constituents of interest upon application of an applied stimulus (e.g., light) include, but are not limited to, benzophenone groups, and the like. Once activated by the applied stimulus, the functional group may bond to the constituent of interest (e.g., protein or peptide) forming a covalent bond between the separation medium and the constituent of interest. For example, the functional group may form a carbon-carbon bond between the functional group and the constituent of interest.

In some embodiments, the functional groups are co-polymerized with the separation medium. For example, the functional groups may include a linker group that is attached to the separation medium. The functional group may be bound to the linker group at a first end of the linker group, and a second end of the linker group may be bound to the separation medium, thereby indirectly bonding the functional group to the separation medium. In some instances, the second end of the linker group, which is bound to the separation medium, includes a co-monomer, such as, but not limited to, an acrylamide co-monomer, and the like. In some embodiments, the second end of the linker group includes a methacrylamide co-monomer. In certain cases, the functional group is a benzophenone functional group and the linker group includes a co-monomer, such as an acrylamide co-monomer. For example, the functional group (including the linker group) may be N-(3-[(4-benzoylphenyl)formamido]propyl) methacrylamide (also known as BPMA or BPMAC) or 3-benzoyl-N-[3-(2-methyl-acryloylamino)-propyl]-benzamide (BP-APMA); the structures of each of which are shown below. Other functional groups that may be used in the separation medium include, but are not limited to, acrylazide and aryl azide functional groups. As described above, the linker group may have the functional group attached at a first end, and the second end of the linker group bound to the polymeric medium. In some instances, the linker group includes a spacer group, such as a spacer group between the first end and the second end of the linker group (e.g., a spacer group in the middle portion of the linker group between the functional group and the co-monomer). In some cases, the spacer group of the linker group between the first and second ends of the linker group includes an aliphatic group, such as, but not limited to, a C₁₋₁₀ alkyl group. In certain cases, the spacer group of the linker group includes a lower alkyl group (e.g., a C₁₋₆ alkyl group, or a C₁₋₅ alkyl group, or a C₁₋₄ alkyl group, or a C₁₋₃ alkyl group, or a C₁₋₂ alkyl group). For instance, the spacer group of the linker group may include a propyl group.

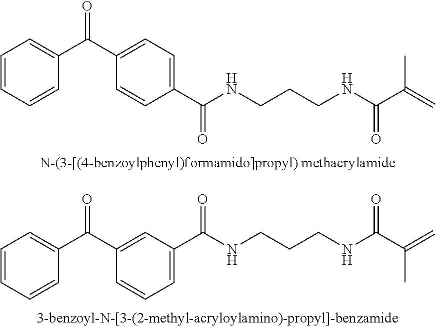 [SMILES: C=C(C)C(=O)NCCCNC(=O)c1ccc(C(=O)c2ccccc2)cc1 ; C=C(C)C(=O)NCCCNC(=O)c1cccc(C(=O)c2ccccc2)c1]

An embodiment of the functional groups that may be co-polymerized with the separation medium is a cross-linked polyacrylamide gel separation medium that includes photoactive benzophenone functional groups. The photoactive benzophenone groups may be activated by light to form covalent bonds to constituents of interest (e.g., proteins in the separated sample).

In certain embodiments, the separation medium is configured to bind to constituents in a sample at a minimum capture efficiency. The capture efficiency is the percentage of constituents in the sample that are bound by the separation medium. In some instances, the capture efficiency, η, is the ratio of fluorescence measured after gradient washout (AFU_{w}) to the fluorescence during focusing (AFU_{f}), corrected by a factor ε to account for the anticipated influence of pH on the species fluorescence signal. In certain embodiments, the separation medium is configured to have a capture efficiency of 1% or more, such as 5% or more, including 10% or more, or 20% or more, or 30% or more, or 40% or more, or 50% or more, or 60% or more, or 70% or more, or 80% or more, or 90% or more, or 95% or more, or 96% or more, or 97% or more, or 98% or more, or 99% or more. In some instances, the separation medium has a capture efficiency of 75% or more.

Additional aspects of the polymeric separation medium are described in U.S. Application Publication No. 2011/0177618, filed May 18, 2010, U.S. Application Publication No. 2012/0329040, filed Jun. 21, 2012, and PCT/US2014/021399, filed Mar. 6, 2014, the disclosures of each of which are incorporated herein by reference.

Additional Aspects of the Devices

In certain embodiments, the separation devices are microfluidic separation devices. A “microfluidic device” is a device that is configured to control and manipulate fluids geometrically constrained to a small scale (e.g., sub-millimeter). Embodiments of the microfluidic devices include a polymeric medium, e.g., a polymeric separation medium as described in more detail herein. The polymeric medium may include a covalently bound capture member that specifically binds to an analyte of interest in a sample. The separation devices of the present disclosure may also be configured to perform assays on a larger scale, such as fluidic device configured to control and manipulate fluids on a millimeter (e.g., milliliter) scale, or larger.

In certain embodiments, the separation device includes a solid support. The solid support may be configured to support a polymeric medium (e.g., the polymeric separation medium). For example, the polymeric separation medium may be provided on the solid support, such that at least a portion of the polymeric separation medium is in contact with a surface of the solid support (e.g., the device includes a solid support carrying the polymeric medium). In some cases, the solid support is composed of a material that is inert (e.g., does not degrade or react) with respect to the samples, buffers, reagents, etc. used in the subject devices and methods. For instance, the solid support may be made of a material, such as, but not limited to, glass, quartz, polymers, elastomers, paper, combinations thereof, and the like. In certain embodiments, the solid support is substantially transparent. By “transparent” is meant that a substance allows visible light to pass through the substance. In some embodiments, a transparent solid support facilitates detection of analytes bound to the polymeric medium, for example analytes that include, produce, or are labeled with a detectable label, such as a fluorescent label. In some cases, the solid support is substantially opaque. By “opaque” is meant that a substance substantially blocks visible light from passing through the substance. In certain instances, an opaque solid support may facilitate the analysis of analytes that are sensitive to light, such as analytes that react or degrade in the presence of light.

In certain embodiments, the solid support is sized to accommodate the polymeric separation medium. For example the solid support may have dimensions (e.g., length and width) such that the entire polymeric separation medium is supported by the solid support. In some cases, the solid support may have dimensions (e.g., length and width) larger than the polymeric separation medium. In some instances, the solid support has dimensions in the range of 10 mm×10 mm to 200 mm×200 mm, including dimensions of 100 mm×100 mm or less, such as 50 mm×50 mm or less, for instance 25 mm×25 mm or less, or 10 mm×10 mm or less, or 5 mm×5 mm or less, for instance, 1 mm×1 mm or less. In some cases, the solid support has a thickness ranging from 0.5 mm to 5 mm, or 1 mm to 4 mm, of 1 mm to 3 mm, or 1 mm to 2 mm. In certain instances, the solid support has a thickness of 1 mm.

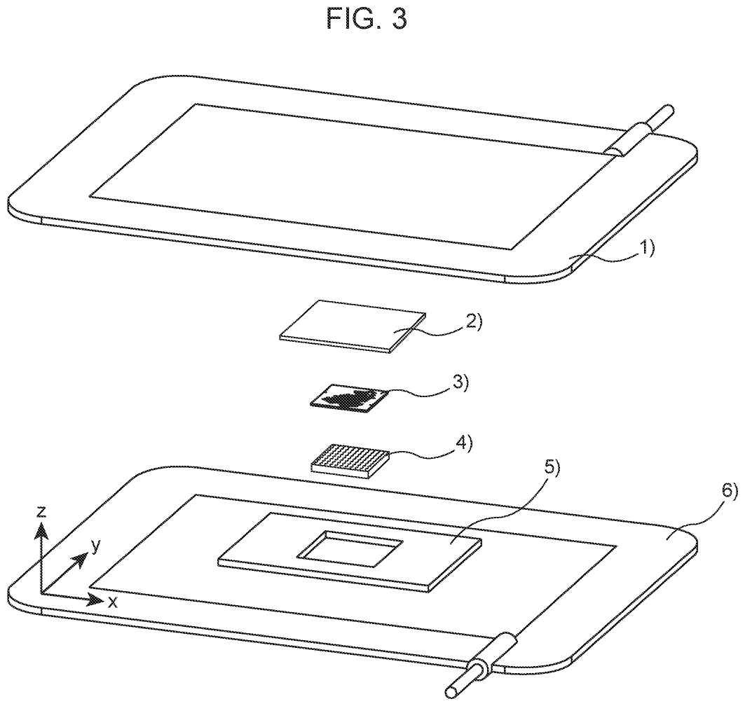

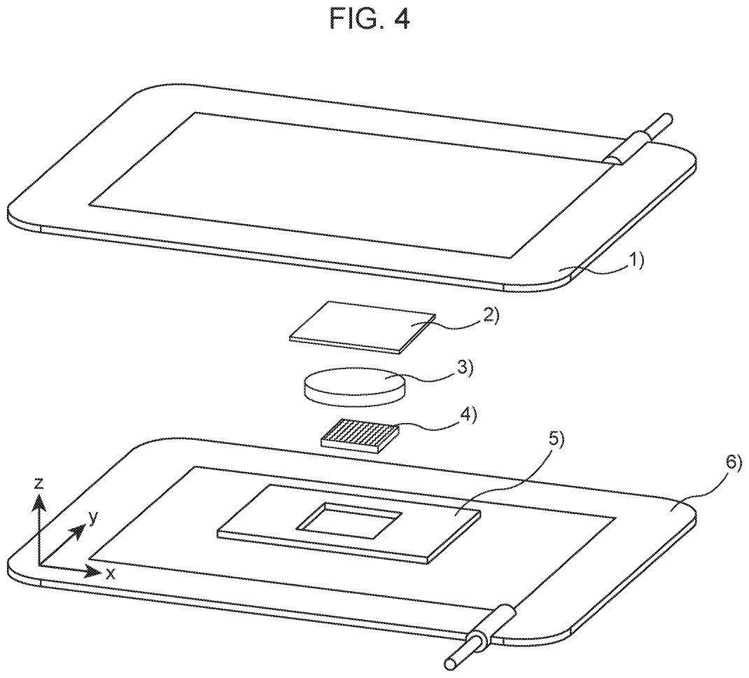

In certain embodiments, the polymeric separation medium is supported on an electrode, for example an electrode used to apply an electric field to the polymeric separation medium during separation of the analytes in the sample. In some cases, the tissue sample is positioned between the electrode and the polymeric separation medium. In addition, a second electrode may be disposed on the opposing surface of the polymeric separation medium (see, e.g., FIG. 3).

In certain embodiments, the tissue sample is supported by a support medium. The support medium may be any convenient support medium, and in some case is a polymeric gel support medium. The composition of the support medium may be the same or different as the composition of the polymeric separation medium. In some instances, the support medium facilitates handling and positioning of the tissue sample, such as during positioning of the tissue sample on the polymeric separation medium.

In certain cases, the tissue sample is disposed on a surface of the support medium, such that the tissue sample is in contact with a surface of the support medium. In these instances, one surface of the tissue sample is in contact with a surface of the polymeric separation medium and an opposing surface of the tissue sample is in contact with the support medium. Stated another way, the tissue sample may be sandwiched in between the support medium and the polymeric separation medium.

In other embodiments, the tissue sample may be contained in the support medium. For instance, the tissue sample may be prepared such that the tissue sample is surrounded or embedded within the support medium. In some instances, a tissue sample contained in a support medium may facilitate handling and positioning of the tissue sample without significant damage to the tissue sample. In these instances, where the tissue sample is contained in a support medium, a surface of the support medium is in contact with a surface of the polymeric separation medium. During analysis, analytes from the tissue sample may traverse through a region of the support medium between the tissue sample and the polymeric separation medium and then enter the polymeric separation medium.

In certain embodiments, the device includes a chamber that contains the tissue sample and the polymeric separation medium (and the support medium, if present). As such, the chamber may have dimensions sufficiently large enough to contain at least the tissue sample and the polymeric separation medium (and the support medium, if present). For example, the chamber may have dimensions greater than the tissue sample and the polymeric separation medium (and the support medium, if present). In some instances, the chamber is water-tight. In some cases, the chamber is air-tight. In some embodiments, the chamber configured to maintain the ambient humidity surrounding the tissue sample and polymeric separation medium, such that the tissue sample and/or polymeric separation medium does not substantially dry out during analysis of the tissue sample.

### Methods

Embodiments of the methods are directed to separating constituents of a tissue sample, such as constituents of cells (e.g., cellular components) from the tissue sample. Aspects of the method include contacting a tissue sample, such as a tissue sample that includes an arrangement of a plurality of associated cells, with a polymeric separation medium. The method also includes applying an electric field to the tissue sample and the polymeric separation medium in a manner sufficient to move one or more analytes from the tissue sample into the polymeric separation medium to separate the analytes in the polymeric separation medium. As described herein, the polymeric separation medium may include functional groups that covalently bond one or more analytes (e.g., cellular components) of interest to the separation medium upon application of an applied stimulus, as described in more detail below. These and other aspects of the methods according to embodiments of the present disclosure are described in the following sections.

Additional aspects of the method may include contacting the tissue sample and the polymeric separation medium with a buffer sufficient to differentially lyse a sub-cellular compartment of cells to produce a set of cellular components. Additional aspects of the method may include contacting the tissue sample and the polymeric separation medium with a buffer sufficient to differentially lyse different cell types in the tissue sample. Additional aspects of the method may include contacting the tissue sample and the polymeric separation medium with a buffer sufficient to either retain or dissociate protein complexes in the tissue sample. Additional aspects of the method may include contacting the tissue sample and the polymeric separation medium with a buffer sufficient for separation of cellular proteins from matrix proteins in the tissue sample.

As described above, methods of the present disclosure include contacting a tissue sample that includes an arrangement of a plurality of associated cells to a polymeric separation medium. In certain embodiments, the tissue sample may be contacted to the polymeric separation medium such that the tissue sample is positioned adjacent to one or more microwells in the polymeric separation medium. For example, the tissue sample may be applied to a surface of the separation medium which has a plurality of microwells.

As described above, the method may include contacting the polymeric separation medium with a buffer sufficient to differentially lyse a sub-cellular compartment of the cell to produce a set of cellular components. By “differentially lyse” or “differential lysis” is meant that the buffer is capable of selectively lysing a specific sub-cellular compartment of the cell without causing significant lysis of other sub-cellular compartment(s) of the cell. For instance, a buffer may be configured to lyse a first sub-cellular compartment, such as the cell membrane, without causing significant lysis of other sub-cellular compartments, such as the nuclear membrane. In some cases, the buffer is configured to selectively lyse the cell membrane such that cytosol is released from the cell without causing significant lysis of other sub-cellular compartments, such as the nuclear membrane. The released cellular components (e.g., cytosol and cellular components contained therein) of the first sub-cellular compartment may then be analyzed (e.g., separated) in the polymeric separation medium. For example, as described herein, the method may include applying an electric field to the polymeric separation medium in a manner sufficient to move at least some of the first set of cellular components (e.g., cytosol components) into the polymeric separation medium to produce a first set of separated cellular components (e.g., a set of separated cytosol components) in the polymeric separation medium. In certain embodiments, the same buffer is sufficient for differentially lysing a sub-cellular compartment and for performing the separation in the polymeric separation medium. Stated another way, the same buffer may be used to lyse the first sub-cellular compartment and also may be used for the electrophoretic separation of the first set of cellular components (e.g., cytosol components) in the polymeric separation medium.

In certain embodiments, the method includes contacting the polymeric separation medium with a second buffer sufficient to differentially lyse a second sub-cellular compartment of the cell to produce a second set of cellular components. For instance, a second buffer may be configured to lyse a second sub-cellular compartment, such as the nuclear membrane. In certain embodiments, the second buffer does not cause significant lysis of other sub-cellular compartments, such as mitochondria, plastids, or other organelles. In some cases, the second buffer is configured to selectively lyse the nuclear membrane such that the contents of the cell nucleus are released from the nucleus without causing significant lysis of other sub-cellular compartments, such as mitochondria, plastids, or other organelles. The released cellular components of the second sub-cellular compartment (e.g., nucleus) may then by analyzed (e.g., separated) in the polymeric separation medium. For example, as described herein, the method may include applying an electric field to the polymeric separation medium in a manner sufficient to move at least some of the second set of cellular components (e.g., nuclear components) into the polymeric separation medium to produce a second set of separated cellular components (e.g., a set of separated nuclear components) in the polymeric separation medium. In certain embodiments, the same buffer is sufficient for differentially lysing a sub-cellular compartment and for performing the separation in the polymeric separation medium. Stated another way, the same buffer may be used to lyse the second sub-cellular compartment and may also be used for the electrophoretic separation of the second set of cellular components (e.g., nuclear components) in the polymeric separation medium. In certain cases, the second buffer is different from the first buffer described above.

In certain instances, the separation of the first set of sub-cellular components is performed in a first polymeric separation medium. In some cases, the separation of the second set of sub-cellular components is performed in a second polymeric separation medium. After separation of the first set of analytes, the first polymeric separation medium may be replaced with the second polymeric separation medium and separation of the second set of analytes may be performed in the second polymeric separation medium.

In certain embodiments, the method includes further lysis of additional sub-cellular compartments in series, such that cellular components of the additional sub-cellular compartments may be analyzed (e.g., separated) in series. The other sub-cellular compartments that may be differentially lysed and analyzed using the methods and devices of the present disclosure may include, but are not limited to, mitochondria, plastids, and other organelles. In certain embodiments, each set of cellular components is separated in different polymeric separation media.

Similarly, the cellular components of different cell types may be separated and analyzed using different polymeric separation media in series. As such, the method may include contacting the polymeric separation medium with a buffer sufficient to differentially lyse a first cell type to produce a set of cellular components. Differential lysis of a first cell type refers to a buffer capable of selectively lysing a first type of cell without causing significant lysis of other types of cells in the tissue sample. The released cellular components (e.g., cytosol and cellular components contained therein) of the first cell type may then be analyzed (e.g., separated) in the polymeric separation medium. For example, as described herein, the method may include applying an electric field to the polymeric separation medium in a manner sufficient to move at least some of the cellular components from the first cell type into the polymeric separation medium to produce a first set of separated cellular components in the polymeric separation medium. In certain embodiments, the same buffer is sufficient for differentially lysing a first cell type and for performing the separation in the polymeric separation medium. Stated another way, the same buffer may be used to lyse the first type of cell and also may be used for the electrophoretic separation of the cellular components from the first cell type in the polymeric separation medium.

In certain embodiments, the method includes contacting the polymeric separation medium with a second buffer sufficient to differentially lyse a second cell type to produce a second set of cellular components. The released cellular components of the second cell type may then by analyzed (e.g., separated) in the polymeric separation medium. For example, as described herein, the method may include applying an electric field to the polymeric separation medium in a manner sufficient to move at least some of the cellular components from the second cell type into the polymeric separation medium to produce a second set of separated cellular components in the polymeric separation medium. In certain embodiments, the same buffer is sufficient for differentially lysing a second cell type and for performing the separation in the polymeric separation medium. Stated another way, the same buffer may be used to lyse the second cell type and may also be used for the electrophoretic separation of the second set of cellular components in the polymeric separation medium. In certain cases, the second buffer is different from the first buffer described above.

In certain instances, the separation of the cellular components from the first cell type is performed in a first polymeric separation medium. In some cases, the separation of the second set of cellular components from the second cell type is performed in a second polymeric separation medium. After separation of the first set of analytes, the first polymeric separation medium may be replaced with the second polymeric separation medium and separation of the second set of analytes may be performed in the second polymeric separation medium.

Similarly, cellular proteins and matrix proteins may be separated and analyzed using different polymeric separation media in series. For example, components of the extracellular matrix and cellular components in the tissue sample may be separated and analyzed using different polymeric separation media in series. As such, the method may include contacting the polymeric separation medium with a buffer sufficient to produce a set of extracellular components. The buffer can be sufficient for releasing extracellular components from the extracellular matrix in the tissue sample and for electrophoretic transport of the extracellular components without causing significant lysis of cells in the tissue sample. The extracellular components released from the tissue sample may then be analyzed (e.g., separated) in the polymeric separation medium. For example, as described herein, the method may include applying an electric field to the polymeric separation medium in a manner sufficient to move at least some of the extracellular components into the polymeric separation medium to produce a set of separated extracellular components in the polymeric separation medium. In certain embodiments, the same buffer is sufficient for releasing the extracellular components from the extracellular matrix and for performing the separation in the polymeric separation medium. Stated another way, the same buffer may be used to release and transport the extracellular components and also may be used for the electrophoretic separation of the extracellular components in the polymeric separation medium.

In certain embodiments, the method includes contacting the polymeric separation medium with a second buffer sufficient to lyse cells to produce a set of cellular components. The cellular components released from cells in the tissue sample may then by analyzed (e.g., separated) in the polymeric separation medium. For example, as described herein, the method may include applying an electric field to the polymeric separation medium in a manner sufficient to move at least some of the cellular components from the cells in the tissue sample into the polymeric separation medium to produce a set of separated cellular components in the polymeric separation medium. In certain embodiments, the same buffer is sufficient for lysing cells in the tissue sample and for performing the separation in the polymeric separation medium. Stated another way, the same buffer may be used to lyse the cells in the tissue sample and may also be used for the electrophoretic separation of the cellular components in the polymeric separation medium. In certain cases, the second buffer is different from the first buffer described above.

In certain instances, the separation of the extracellular components from the extracellular matrix is performed in a first polymeric separation medium. In some cases, the separation of the set of cellular components from the cells in the tissue sample is performed in a second polymeric separation medium. After separation of the first set of analytes, the first polymeric separation medium may be replaced with the second polymeric separation medium and separation of the second set of analytes may be performed in the second polymeric separation medium.

In certain embodiments, the method may further include separating the analytes from the tissue sample in the separation medium to produce separated sample constituents. In some cases, the separated constituents are produced by gel electrophoresis as the analytes traverse through the separation medium. In other cases, the separated analytes are produced by isoelectric focusing in the separation medium. The separated analytes may include distinct detectable bands or zones of constituents (e.g., analytes), where each band or zone includes one or more constituents that have substantially similar properties, such as molecular mass, size, charge (e.g., charge to mass ratio), isoelectric point, affinity interaction, etc. depending on the type of separation performed.

For example, in embodiments where the polymeric separation medium includes a planar array of microwells as described herein, the method may include separating the sample constituents by applying an electric field across the polymeric separation medium in a manner sufficient to move at least some of the sample constituents from the microwell and into the polymeric separation medium to produce separated sample constituents in the polymeric separation medium as the sample constituents traverse through the separation medium.

In certain embodiments, the method includes applying an electric field to the tissue sample and the polymeric separation medium. The electric field may facilitate the movement of the sample constituents through the device (e.g., electrokinetic transfer of the sample from one region of the device to another region of the device). The electric field may also facilitate the separation of the analytes in the sample by electrophoresis (e.g., polyacrylamide gel electrophoresis (PAGE), SDS-PAGE, isoelectric focusing, etc.), as described above.

For instance, separating the analytes in a sample may include applying an electric field configured to direct the analytes in the sample through the separation medium of the device. The electric field may be configured to facilitate the separation of the analytes in a sample based on the physical properties of the analytes. For example, the electric field may be configured to facilitate the separation of the analytes in the sample based on the molecular mass, size, charge (e.g., charge to mass ratio), isoelectric point, etc. of the analytes. In certain instances, the electric field is configured to facilitate the separation of the analytes in the sample based on the molecular mass of the analytes. In other embodiments, the electric field is configured to facilitate separation of the analytes in the sample based on the isoelectric point (pl) of the analytes.

In some instances, the method further includes immobilizing the separated sample components in the polymeric separation medium. Immobilizing may be accomplished using any convenient approach, e.g., covalently bonding the separated sample components to the polymeric separation medium, such as by exposing the polymeric separation medium to an applied stimulus to immobilize the separated analytes in the polymeric separation medium. In some instances, the applied stimulus is ultraviolet (UV) light. For example, after the constituents in the sample have been separated, the method may further include applying a stimulus to the separation medium to covalently bond the constituents to the separation medium. In some cases, the applying the stimulus includes applying electromagnetic radiation to the separation medium. For instance, the method may include exposing the separation medium to light, such as, but not limited to, visible light, UV light, infrared light, etc. In certain cases, the method includes applying light (e.g., UV light) to the separation medium to covalently bond the constituents to the separation medium.

As such, in certain embodiments, the method includes immobilizing the set of separated cellular components in the polymeric separation medium as described above. The set of separated cellular components may be the set of cellular components produced by differential lysis of a sub-cellular compartment (e.g., cytosol) as described above. In some instances, following immobilization of a first set of cellular components in a first polymeric separation medium, a second polymeric separation medium is contacted with a second buffer sufficient to differentially lyse a second sub-cellular compartment of the cells to produce a second set of cellular components, as described above. The second set of cellular components may be analyzed (e.g., separated) in the second polymeric separation medium as described above. In some cases, the method further includes immobilizing the second set of separated cellular components in the second polymeric separation medium. The second set of separated cellular components may be the set of cellular components produced by differential lysis of a sub-cellular compartment (e.g., nucleus) as described above.

Similarly, the set of separated cellular components may be the set of cellular components produced by differential lysis of different cell types, as described above. For example, the method may include immobilizing the first set of separated cellular components from the first cell type in the polymeric separation medium, as described above, followed by separation and immobilization of the second set of separated cellular components from the second cell type in the polymeric separation medium. Similarly, the sets of separated components may include a set of extracellular components produced from an extracellular matrix and a set of cellular components from cells in a tissue sample, as described above. For example, the method may include immobilizing the set of separated extracellular components from the extracellular matrix in the polymeric separation medium, as described above, followed by separation and immobilization of the set of separated cellular components from the cells in the polymeric separation medium.

In certain embodiments, the light used to covalently bond the constituents of interest to the separation medium has a wavelength different from the light used to activate formation of the separation medium. For example, as described herein, the light used to activate formation of the separation medium may have a wavelength of blue light in the visible spectrum. As described above, the light used to covalently bond the constituents of interest to the separation medium may have a wavelength of UV light. As such, in certain embodiments, the method includes exposing the separation medium to a first wavelength of light to form the separation medium, and exposing the separation medium to a second wavelength of light to covalently bond the constituents of interest to the separation medium. The first and second wavelengths of light may be blue light and UV light, respectively, as described herein.

In certain embodiments, the method includes determining whether an analyte of interest is present in a sample, e.g., determining the presence or absence of one or more analytes of interest in a sample. In some instances, the devices are configured to detect the presence of one or more analytes in a sample. In certain embodiments of the methods, the presence of one or more analytes in the sample may be determined qualitatively or quantitatively. Qualitative determination includes determinations in which a simple yes/no result with respect to the presence of an analyte in the sample is provided to a user. Quantitative determination includes both semi-quantitative determinations in which a rough scale result, e.g., low, medium, high, is provided to a user regarding the amount of analyte in the sample and fine scale results in which a numerical measurement of the concentration of the analyte is provided to the user.

In certain embodiments, the method includes detecting an analyte of interest bound to the separation medium. Detectable binding of an analyte of interest to the separation medium indicates the presence of the analyte of interest in the sample. In some instances, detecting the analyte of interest includes contacting the analyte of interest with a label configured to specifically bind to the analyte of interest, e.g., as may be present in an analyte detection reagent. The analyte detection reagent can be any molecule that specifically binds to a protein or nucleic acid sequence or biomacromolecule that is being targeted (e.g., the analyte of interest). Depending on the nature of the analyte, the analyte detection reagent can be, but is not limited to: single strands of DNA complementary to a unique region of the target DNA or RNA sequence for the detection of nucleic acids; antibodies against an epitope of a peptidic analyte for the detection of proteins and peptides; or any recognition molecule, such as a member of a specific binding pair. For example, suitable specific binding pairs include, but are not limited to: a member of a receptor/ligand pair; a ligand-binding portion of a receptor; a member of an antibody/antigen pair; an antigen-binding fragment of an antibody; a hapten; a member of a lectin/carbohydrate pair; a member of an enzyme/substrate pair; biotin/avidin; biotin/streptavidin; digoxin/antidigoxin; a member of a DNA or RNA aptamer binding pair; a member of a peptide aptamer binding pair; and the like. In certain embodiments, the analyte detection reagent includes an antibody. The antibody may specifically bind to the analyte of interest.

In certain embodiments, the analyte detection reagent includes a detectable label. Detectable labels include any convenient label that may be detected using the methods and systems, and may include, but are not limited to, fluorescent labels, colorimetric labels, chemiluminescent labels, multicolor reagents, enzyme-linked reagents, avidin-streptavidin associated detection reagents, radiolabels, gold particles, magnetic labels, and the like. In certain embodiments, the analyte detection reagent includes an antibody associated with a detectable label. For example, the analyte detection reagent may include a labeled antibody (e.g., a fluorescently labeled antibody) that specifically binds to the analyte of interest. As such, the method may include detecting the labeled analyte of interest.

As described above, detecting the analyte of interest includes contacting the analyte of interest with an analyte detection reagent (e.g., a label) configured to specifically bind to the analyte of interest (e.g., an antibody that specifically binds to the analyte of interest). For example, contacting the analyte of interest with an analyte detection reagent may include applying a solution of analyte detection reagent to the polymeric separation medium. The analyte detection reagent may be contacted to any surface of the polymeric separation medium, such as the top or one or more sides of the polymeric separation medium. In some cases, the analyte detection reagent may be moved through the polymeric separation medium such that the analyte detection reagent contacts analytes of interest immobilized within the polymeric separation medium. For instance, the analyte detection reagent may be moved through the polymeric separation medium by applying an electric field to the polymeric separation medium, applying a pressure, applying a centrifugal force, passive diffusion, and the like.

In certain embodiments, detecting the analyte of interest includes contacting the analyte of interest with a primary label that specifically binds to the analyte of interest. In certain embodiments, the method includes enhancing the detectable signal from the labeled analyte of interest. For instance, enhancing the detectable signal from the labeled analyte of interest may include contacting the primary label with a secondary label configured to specifically bind to the primary label. In certain instances, the primary label is a primary antibody that specifically binds to the analyte of interest, and the secondary label is a secondary antibody that specifically binds to the primary antibody. As such, enhancing the detectable signal from the labeled analyte of interest may include contacting the primary antibody with a secondary antibody configured to specifically bind to the primary antibody. The use of two or more detectable labels as described above may facilitate the detection of the analyte of interest by improving the signal-to-noise ratio.

In certain embodiments, the analyte detection reagent may not specifically bind to an analyte of interest. In some cases, the analyte detection reagent may be configured to produce a detectable signal from the analyte of interest without specifically binding to the analyte of interest. For example, the analyte of interest may be an enzyme (e.g., a cellular enzyme) and the analyte detection reagent may be a substrate for the enzyme. In some cases, contacting the analyte detection reagent (e.g., enzyme substrate) to the analyte of interest (e.g., enzyme) may produce a detectable signal as the substrate is converted by the enzyme.

In certain embodiments, the method includes removing the analyte detection reagent and then contacting the analyte of interest with another analyte detection reagent (e.g., stripping and reprobing). For instance, the method may include contacting the labeled analyte of interest with a buffer (e.g., a stripping buffer) configured to dissociate the analyte detection reagent from the analyte of interest. The dissociated analyte detection reagent may then be washed from the polymeric separation medium. In some cases, the analyte of interest may then be contacted with a subsequent analyte detection reagent. The subsequent analyte detection reagent may be the same or different from the initial analyte detection reagent. Stripping and reprobing may facilitate contacting analytes of interest with different analyte detection reagents.

Detection of the separated analytes bound to the polymeric separation medium may facilitate the production of a map of the distribution of the analytes in the tissue sample. As such, aspects of the present methods include detecting the analytes immobilized in the separation medium to produce a map of the distribution of the analytes in the tissue sample. For example, the method may include producing of a map, such as a two-dimensional (2D) or three-dimensional (3D) map, of the relative distribution of analytes in two or three dimensions in a tissue sample. In some instances, when an electric field is applied to the tissue sample and the polymeric separation medium to transport analytes from the tissue sample through the polymeric separation medium, the relative positions of the analytes in the tissue sample remain substantially the same. As such, when the separated analytes are immobilized (bound) to the separation medium as described herein, the immobilization of the separated analytes maintains the separation of the analytes produced by transport of the analytes through the polymeric separation medium (e.g., maintains the separation of the analytes produced by electrophoretic separation), as well as maintains the relative 2D or 3D distribution of the analytes in the context of the tissue sample. Thus, embodiments of the present methods maintain tissue context information of the analytes in the tissue sample, such as but not limited to, position within the tissue sample, proximity to certain features in the tissue sample (e.g., blood vessels), relative composition of different areas in the tissue sample, as well as proximity and composition of neighboring cell types in the tissue sample). Thus, in certain instances, the polymeric separation medium includes one or more separated analytes from the tissue sample, where the separated analytes in the polymeric separation medium are correlated to a distribution of the analytes in the tissue sample. For example, the relative distribution of the analytes in the tissue sample in the x-y plane of the tissue sample can be maintained after separation and immobilization of the analytes in the separation medium. In some cases, the relative distribution of the analytes in the tissue sample in three dimensions can be maintained after separation and immobilization of the analytes in the separation medium.

Analysis of the relative position and composition of the separated analytes in the polymeric separation medium facilitates a determination of the distribution of the analytes in the tissue sample (e.g., a determination of the distribution of the analytes in the tissue sample before analysis). As such, in certain embodiments, the method includes determining the position of one or more analytes in the tissue sample. In some cases, the method includes determining the proximity of one or more analytes to another feature in the tissue sample, where such features may include, but are not limited to, a blood vessel, a tumor, a certain cell type, an organ, etc. In some instances, the method includes determining the relative analyte composition of one or more areas of the tissue sample. In certain embodiments, the method includes determining the proximity and/or composition of one or more areas of different cell types in the tissue sample. In certain embodiments, the method includes determining the relative analyte composition of one or more areas of the extracellular matrix in a tissue sample. In certain embodiments, the method includes determining the position and composition of analytes in one or more areas of the extracellular matrix in a tissue sample relative to the position and composition of analytes from cells in the tissue sample.

In certain embodiments, the method provides a distribution map of analytes in a tissue sample with a spatial resolution of 100 μm or less, such as 90 μm or less, or 80 μm or less, or 70 μm or less, or 60 μm or less, or 50 μm or less, or 40 μm or less, or 30 μm or less, or 20 μm or less, or 10 μm or less, or 5 μm or less, or 1 μm or less. In some instances, the method provides a distribution map of analytes in a tissue sample with a spatial resolution of 50 μm or less.

In certain embodiments, detecting the analyte of interest includes imaging the polymeric separation medium to produce an image of the separated analytes in the polymeric separation medium. Imaging the analytes in the polymeric separation medium may be performed using any convenient imaging device, such as, but not limited to, a camera, a UV detector, a fluorescent detector, combinations thereof, and the like.

In certain embodiments, the method includes storing the polymeric separation medium. For example, the method may include storing the polymeric separation medium by dehydrating the polymeric separation medium. The polymeric separation medium may be stored for an extended period of time, such as, but not limited to, 1 day, 2 days, 3 days, 4 days, 5 days, 6 days, 1 week, 2 weeks, 3 weeks, 1 month, 2 months, 3 months, 4 months, 5 months, 6 months or more. In some embodiments, the method further includes rehydrating the polymeric separation medium. The rehydrated polymeric separation medium may be used in any of the assay steps described herein. For example, dehydrating and rehydrating the polymeric separation medium may be performed between any of the assay steps, such as, between producing the polymeric separation medium and performing an assay, between immobilizing the analytes of interest to the polymeric separation medium and contacting the analytes with an analyte detection reagent, between stripping and reprobing, etc.

Samples that may be assayed with the subject methods may include both simple and complex samples. Simple samples are samples that include the analyte of interest, and may or may not include one or more molecular entities that are not of interest, where the number of these non-interest molecular entities may be low, e.g., 10 or less, 5 or less, etc. Simple samples may include initial biological or other samples that have been processed in some manner, e.g., to remove potentially interfering molecular entities from the sample. By “complex sample” is meant a sample that may or may not have the analyte of interest, but also includes many different proteins and other molecules that are not of interest. In some instances, the complex sample assayed in the subject methods is one that includes 10 or more, such as 20 or more, including 100 or more, e.g., 10³ or more, 10⁴ or more (such as 15,000; 20,000 or 25,000 or more) distinct (i.e., different) molecular entities, that differ from each other in terms of molecular structure or physical properties (e.g., molecular mass, size, charge, isoelectric point, affinity interaction, etc.). In some embodiments, the sample is a complex sample, such as a tissue sample, such as a tissue slice (see, e.g., FIG. 3). In some cases, the sample is a purified protein sample (see, e.g., FIG. 4).

In certain embodiments, the analytes of interest are cells and/or cellular components. In some cases, the cells are obtained from tissue samples (e.g., biological tissue samples), such as but not limited to, a tissue slice, a biopsy tissue, and the like. In certain instances, the sample is a sample, such as a purified protein sample, that was isolated from a biological source, such as, but not limited to, urine, blood, serum, plasma, saliva, semen, prostatic fluid, nipple aspirate fluid, lachrymal fluid, perspiration, feces, cheek swabs, cerebrospinal fluid, cell lysate samples, amniotic fluid, gastrointestinal fluid, and the like. The sample can be a biological sample or can be extracted from a biological sample derived from humans, animals, plants, fungi, yeast, bacteria, tissue cultures, viral cultures, or combinations thereof using conventional methods for the successful extraction of DNA, RNA, proteins and peptides. In certain embodiments, the sample is a fluid (e.g., liquid) sample, such as a solution of analytes (e.g., cells and/or cellular components) in a fluid. The fluid may be an aqueous fluid, such as, but not limited to water, a buffer, and the like.

As described above, the samples that may be assayed in the subject methods may include one or more analytes of interest. Examples of detectable analytes include, but are not limited to: nucleic acids, e.g., double or single-stranded DNA, double or single-stranded RNA, DNA-RNA hybrids, DNA aptamers, RNA aptamers, etc.; proteins and peptides, with or without modifications, e.g., antibodies, diabodies, Fab fragments, DNA or RNA binding proteins, phosphorylated proteins (phosphoproteomics), peptide aptamers, epitopes, and the like; small molecules such as inhibitors, activators, ligands, etc.; oligo or polysaccharides; mixtures thereof; and the like.

In certain embodiments, the method includes contacting the separated analytes bound to the separation medium with a blocking reagent prior to detecting the analyte of interest. In some cases, contacting the separated analytes with a blocking reagent prior to detecting the analyte of interest may facilitate a minimization in non-specific binding of a detectable label to the separated analytes. For example, contacting the separated analytes with the blocking reagent prior to detecting the analyte of interest may facilitate a minimization in non-specific binding of a labeled antibody to the separated analytes. The blocking reagent can be any blocking reagent that functions as described above, and may include, but is not limited to, bovine serum albumin (BSA), non-fat dry milk, casein, and gelatin. In other embodiments, no blocking step is required. Thus, in these embodiments, the method does not include a blocking step prior to detecting the analyte of interest.

In certain embodiments, the method also includes optional washing steps, which may be performed at various times before, during and after the other steps in the method. For example, a washing step may be performed after binding the separated sample to the separation medium, after contacting the separated sample with the blocking reagent, after contacting the separated sample with the detectable label, etc.

Embodiments of the method may also include releasing the analyte bound to the separation medium. The releasing may include contacting the bound analyte with a releasing agent. The releasing agent may be configured to disrupt the binding interaction between the analyte and the separation medium. In some cases, the releasing agent is a reagent, buffer, or the like, that disrupts the binding interaction between the analyte and the separation medium causing the separation medium to release the analyte. After releasing the analyte from the separation medium, the method may include transferring the analyte away from the separation medium. For example, the method may include directing the released analyte downstream from the separation medium for secondary analysis with a secondary analysis device such as, but is not limited to, a UV spectrometer, and IR spectrometer, a mass spectrometer, an HPLC, an affinity assay device, a second microfluidic device as described herein, and the like.

In some embodiments, the methods include the uniplex analysis of an analyte in a sample. By “uniplex analysis” is meant that a sample is analyzed to detect the presence of one analyte in the sample. For example, a tissue sample may include an analyte of interest and other molecular entities that are not of interest. In some cases, the methods include the uniplex analysis of the tissue sample to determine the presence and/or distribution of the analyte of interest in the tissue sample.

Certain embodiments include the multiplex analysis of two or more analytes in a sample. By “multiplex analysis” is meant that the presence and/or distribution of two or more distinct analytes, in which the two or more analytes are different from each other, is determined. For example, analytes may include detectable differences in their molecular mass, size, charge (e.g., mass to charge ratio), isoelectric point, and the like. In some instances, the number of analytes is greater than 2, such as 4 or more, 6 or more, 8 or more, etc., up to 20 or more, e.g., 50 or more, including 100 or more, distinct analytes. In certain embodiments, the methods include the multiplex analysis of 2 to 100 distinct analytes, such as 4 to 50 distinct analytes, including 4 to 20 distinct analytes. In certain embodiments, multiplex analysis also includes the use of two or more different detectable labels. The two or more different detectable labels may specifically bind to the same or different analytes. In some cases, the two or more different detectable labels may specifically bind to the same analyte. For instance, the two or more different detectable labels may include different antibodies specific for different epitopes on the same analyte. The use of two or more detectable labels specific for the same analyte may facilitate the detection of the analyte by improving the signal-to-noise ratio. In other cases, the two or more different detectable labels may specifically bind to different analytes. For example, the two or more detectable labels may include different antibodies specific for epitopes on different analytes. The use of two or more detectable labels each specific for different analytes may facilitate the detection of two or more respective analytes in the sample in a single assay.

In certain embodiments, the method is an automated method. As such, the method may include a minimum of user interaction with the devices and systems after introducing the sample into the device. For example, the steps of separating the sample constituents in the separation medium to produce a separated sample and applying the stimulus to the separation medium to covalently bond the constituents to the separation medium may be performed by the device and system at predetermined intervals, such that the user need not manually perform these steps. In some cases, the automated method may facilitate a reduction in the total assay time. For example, embodiments of the method, including the separation and detection of analytes in a sample, may be performed in 240 minutes or less, e.g., 180 minutes or less, 120 minutes or less, such as 90 minutes or less, or 60 minutes or less, or 45 minutes or less, or 30 minutes or less, such as 20 minutes or less, including 15 minutes or less, or 10 minutes or less, or 5 minutes or less, or 2 minutes or less, or 1 minute or less.

Aspects of embodiments of the present disclosure further include methods of making the above-described polymeric separation medium. In some instances, the methods include positioning a monomeric precursor composition of the polymeric separation medium between a first surface and second surface comprising one or more structural features; irradiating the monomeric precursor composition with light having a wavelength sufficient (e.g., blue light) to initiate polymerization of the precursor composition so as to produce the desired composition. The method may further include removing the second surface comprising the one or more structural features such that the first surface (e.g., the solid support) carries a polymeric separation medium that includes a plurality of microwells as described herein. In certain embodiments, the structural features on the second surface include a plurality of posts. The posts on the second surface may include shapes and sizes that correspond to the desired shapes and sizes of the interior volumes of the microwells. In embodiments that include a plurality of posts on the second surface, a polymeric separation medium may be produced that includes a planar array of microwells.

In certain embodiments, the subject method includes assembling a device for performing the method of analyte analysis described herein. Assembling the device may include disposing a tissue sample on a support, such as on a first electrode. As described herein the tissue sample can be disposed on a surface of a support medium to facilitate positioning of the tissue sample on the support (e.g., first electrode). In other instances described herein, the tissue sample is contained in a support medium, and a surface of the support medium is contacted with the support (e.g., first electrode). The method of assembling the device may also include positioning a polymeric separation medium in fluid communication with the tissue sample. A surface of the polymeric separation medium may be placed in contact with a surface of the tissue sample (e.g., in embodiments where the tissue sample is provided on a surface of a support medium). In other cases, a surface of the polymeric separation medium is placed in contact with a surface of the support medium (e.g., in embodiments where the tissue sample is contained in the support medium. As described herein, the surface of the polymeric separation medium having an array of microwells may be the surface in contact with the tissue sample or the support medium containing the tissue sample. In some cases, the array of microwells facilitates a reduction in the diffusion of sample constituents in the x-y plane. In certain cases, assembling the device also includes disposing a second electrode on an opposing surface of the polymeric separation medium; i.e., the surface opposite the surface in fluid communication with the tissue sample.

### Systems

Aspects of certain embodiments include a system configured to perform methods of the present disclosure. In some instances, the system includes a device containing a polymeric separation medium as described herein. In certain embodiments, the system includes a buffer.

The system may also include a source of electromagnetic radiation (i.e., an electromagnetic radiation source). In some cases, the electromagnetic radiation source is a light source. For example, the light source may include a visible light source, a UV light source, an infrared light source, etc. In some instances, the electromagnetic radiation source includes a light source, such as a UV light source. As described above, the electromagnetic radiation source may be used to apply electromagnetic radiation to the separation medium in the microfluidic device to immobilize (e.g., covalently bond) sample constituents to the separation medium.

In certain embodiments, the system also includes a detector. In some cases, the detector is configured to detect a detectable label. The detector may include any type of detector configured to detect the detectable label used in the assay. As described above, detectable label may be a fluorescent label, colorimetric label, chemiluminescent label, multicolor reagent, enzyme-linked reagent, avidin-streptavidin associated detection reagent, radiolabel, gold particle, magnetic label, etc. In some instances, the detectable label is a fluorescent label. In these instances, the detector may be configured to contact the fluorescent label with electromagnetic radiation (e.g., visible, UV, x-ray, etc.), which excites the fluorescent label and causes the fluorescent label to emit detectable electromagnetic radiation (e.g., visible light, etc.). The emitted electromagnetic radiation may be detected by the detector to determine the presence of the labeled analyte bound to the separation medium.

In some instances, the detector may be configured to detect emissions from a fluorescent label, as described above. In certain cases, the detector includes a photomultiplier tube (PMT), a charge-coupled device (CCD), an intensified charge-coupled device (ICCD), a complementary metal-oxide-semiconductor (CMOS) sensor, a visual colorimetric readout, a photodiode, and the like. The system may be configured to produce an image of the separated cellular components based on a signal obtained from the detector.

Systems of the present disclosure may include various other components as desired. For example, the systems may include fluid handling components. The fluid handling components may be configured to direct one or more fluids through the device. In some instances, the fluid handling components are configured to direct fluids, such as, but not limited to, fluid samples, buffers (e.g., electrophoresis buffers, lysis buffers, electrophoresis/lysis buffers, wash buffers, release buffers, etc.), and the like. In certain embodiments, the fluid handling components are configured to deliver a fluid to the separation medium of the device, such that the fluid contacts the separation medium. The fluid handling components may include pumps. In some cases, the pumps are configured for pressure-driven fluid handling and routing of fluids through the devices and systems disclosed herein.

In certain embodiments, the systems include one or more electric field generators. An electric field generator may be configured to apply an electric field to various regions of the device, e.g., to the tissue sample and the separation medium. The system may be configured to apply an electric field such that the sample is electrokinetically transported through the device. For example, the electric field generator may be configured to apply an electric field to the separation medium. In some cases, the applied electric field may be aligned with the directional separation axis of the separation medium. As such, the applied electric field may be configured to electrokinetically transport the analytes and components in a sample through the separation medium. In some instances, the electric field generators are configured to apply an electric field with a strength ranging from 10 V/cm to 1000 V/cm, such as from 100 V/cm to 800 V/cm, including from 200 V/cm to 800 V/cm, or from 400 v/cm to 800 V/cm.

In certain embodiments, the system includes an electric field generator configured to apply an electric field such that analytes and/or constituents in the sample are isoelectrically focused in the separation medium. For instance, an applied electric field may be aligned with the directional axis of the separation medium and configured to isoelectrically focus the sample constituents along the directional axis of the separation medium.

In some embodiments, the electric field may be directionally distinct. For example, the electric field may be aligned with the directional separation axis of the separation medium. The electric field may be configured to direct the sample or analytes through the separation medium along the directional separation axis of the separation medium.

In certain embodiments, the system includes one or more electric field generators configured to generate an electric field. In certain instances, the electric field generators may be proximal to the device, such as arranged on the device. In some cases, the electric field generators are positioned a distance away from the device. For example, the electric field generators may be incorporated into the system for use with the device.

### Utility

The subject devices, systems and methods find use in a variety of different applications where determination of the presence or absence, and/or quantification of one or more analytes in a tissue sample is desired. For example, the subject devices, systems and methods find use in the separation and detection of proteins, peptides, nucleic acids, and the like, which may be present in a cell or a sub-cellular compartment in a tissue sample. In some cases, the subject devices, systems and methods find use in the separation and detection of cellular proteins. For example, the subject devices, systems and methods find use in the detection of proteins associated with the development of cancer treatments, for development of stem cell therapy, for high-throughput drug screening, for biological analyses regarding human aging, and the like. Embodiments of the subject systems, devices and methods also find use in determining the distribution of analytes in a tissue sample, such as for producing a map (e.g., a 2D or 3D map) of the distribution of analytes in a tissue sample, as described herein.

For example, the subject systems, devices and methods find use in the measurement of proteomic information (including proteoforms and complexes lacking specific antibody probes) alongside tissue context information (e.g., position within tumor, proximity to blood vessels, ECM composition, and neighboring cell types as well as their proximity and proteomic information) using standard immunohistochemistry (IHC) antibodies. This combination measurement finds use in determining the relationships between the tumor microenvironment and molecular information in heterogeneous tumors, facilitating classification by combining molecular (including proteoform and protein complex) and morphologic information to identify enhanced biomarker signatures. This type of measurement will also facilitate a determination of how the tumor microenvironment may affect expression of proteoforms, such as truncated oncoprotein isoforms, which in turn may facilitate diagnostic and treatment methods. For instance, the subject systems, devices and methods find use both in research (e.g., understanding how aspects of the cellular microenvironment can control proteoform expression and protein complex formation) and in diagnostics and pathology (e.g., identifying protein biomarker signatures for diagnosing disease states and selecting appropriate treatments).

The subject systems, devices and methods find use in measuring proteoforms: (1) for which specific antibodies do not exist, (2) that are expressed at or near median abundance (e.g., about 150×10⁶ molecules was the limit of detection for MALDI-FT-ICR, which for single-cell expression likely encompasses <0.1% of the mammalian proteome), or that have higher molecular weight (>70 kDa can be challenging to detect via top-down mass spec, whereas identification of proteoforms via bottom-up mass spec can be challenging because of challenges in tying peptides with low abundance back to specific proteoforms), (3) at a single-cell resolution (or 50 μm), while preserving tissue context information.

The subject devices, systems and methods find use in development and validation of stem cell de-differentiation and differentiation protocols. For instance, induced pluripotent stem cells may be derived from somatic cells such as skin cells, which may involve reprogramming of somatic cells with various external stimuli (e.g., chemical or biological stimuli) to induce the cells to a pluripotent state. In some instances, when experimenting with new external stimuli to achieve pluripotency, it may be desirable to measure the response of the cell population to determine if pluripotency has been achieved. The subject devices, systems and methods find use in measuring these responses of the cell population to determine if pluripotency has been achieved. For example, the subject devices, systems and methods find use in measuring multiple protein targets that are known pluripotency indicators such as, but not limited to, Oct-3/4, Nanog, SSEA-4, and SOX2. The subject devices, systems and methods find use in determining the heterogeneity of the transformed cell population to determine the percentage of the cells that have been successfully transformed to a pluripotent state. Such induced pluripotent stem cells can then be differentiated via external chemical or biological stimuli to derive various cell types such as, but not limited to, cardiomyocytes, neurons, hepatocytes and endothelial cells. The subject devices, systems and methods find use in the validation of such differentiation protocols because, in certain embodiments, subject devices, systems and methods can simultaneously detect multiple protein markers that are indicative of successful differentiation to the target cell type. The subject devices, systems and methods find use in determining the heterogeneity of the transformed cell population to determine the percentage of the cells that have successfully differentiated to the target cell type.

The subject devices, systems and methods also find use in development and validation of “disease-in-a-dish” models. For example, it may be challenging for researchers to study diseases in the human brain since extracting neurons from living patients is difficult and risky. As an alternative, cellular models of disease may be created to allow basic scientific research and drug development. Such models can be created, for example, by differentiation of neurons from induced pluripotent stem cells (IPSCs) derived from skin cells donated by patients with a genetic neurodegenerative disease. To create these models, stem cell differentiation protocols may be developed and validated as previously described to de-differentiate skin cells to stem cells and then differentiate the stem cells to neurons. Once this transformation is successful, the model may be validated by determining that characteristics of the disease are present in the differentiated cells. For example, neurons can be created from the skin cells of patients with Huntington's disease. Once created, the derived cells may be tested for expression of the diseased form of the Huntingtin protein. The subject devices, systems and methods find use in detecting the presence and heterogeneity of the Huntingtin protein in the disease model and verifying similarity to primary cells. Disease-in-a-dish models may also be created through selection or genetic modification of cell lines. Such cells may be validated to ensure that the genetic modification results in stable expression of a diseased biomarker (e.g., a protein) that mimics what is seen in diseased primary cells. The subject devices, systems and methods find use in creating disease models of the liver, kidney, heart, brain, blood or other organs, tissues and cell types.

The subject devices, systems and methods also find use in measuring the heterogeneity of cancerous tumors. Specific biomarkers such as, for example, HER-2 and BRAF, are indicative of certain cancer mutations and are targets for drugs such as trastuzumab and vemurafenib, respectively. Cancer may be a highly heterogeneous disease and targets such as HER-2 and BRAF may not be expressed uniformly within a tumor. Such heterogeneity may have implications for clinical diagnosis and treatment. The subject devices, systems and methods find use in analyzing the heterogeneity of multiple targets in a cell population derived from a tumor biopsy. Such an approach may facilitate basic scientific research, drug discovery and development, and companion diagnostics for targeted therapeutics.

The subject devices, systems and methods also find use in the determination of the mechanism of action of drug compounds. For example, “disease-in-a-dish” models may be used as in vitro test platforms for drug development. Drugs can be developed to target specific cellular targets and pathways that are present in both the disease and disease models. The subject devices, systems and methods find use in analyzing the heterogeneous response of a cell population after exposure to a drug candidate. Response to the drug can be correlated to the presence of the primary target and heterogeneous responses within the cell population of a tissue sample not explained by the presence or absence of the primary target can be further correlated with other proteins and signaling pathways. In this way, the subject devices, systems and methods find use in determining the mechanism of action of the drug, which may facilitate more efficient research, development and eventual approval of the drug compound.

The subject devices, systems and methods find use in the detection of nucleic acids, proteins, or other biomolecules in a tissue sample. The methods may include the detection of a set of biomarkers, e.g., two or more distinct protein biomarkers, in a tissue sample. For example, the methods may be used in the rapid, clinical detection of two or more disease biomarkers in a biological sample, e.g., as may be employed in the diagnosis of a disease condition in a subject, or in the ongoing management or treatment of a disease condition in a subject, etc. In addition, the subject devices, systems and methods may find use in protocols for the detection of an analyte in a tissue sample, such as, but not limited to, Western blotting, and the like.

The subject devices, systems and methods find use in detecting biomarkers. In some cases, the subject devices, systems and methods may be used to detect the presence or absence and/or distribution of particular biomarkers in a tissue sample, as well as an increase or decrease in the concentration or distribution of particular biomarkers in a tissue sample.

The presence or absence of a biomarker or significant changes in the concentration or distribution of a biomarker can be used to diagnose disease risk, presence of disease in an individual, or to tailor treatments for the disease in an individual. For example, the presence or distribution of a particular biomarker or panel of biomarkers may influence the choices of drug treatment or administration regimes given to an individual. In evaluating potential drug therapies, a biomarker may be used as a surrogate for a natural endpoint such as survival or irreversible morbidity. If a treatment alters the biomarker, which has a direct connection to improved health, the biomarker can serve as a surrogate endpoint for evaluating the clinical benefit of a particular treatment or administration regime. Thus, personalized diagnosis and treatment based on the particular biomarkers or panel of biomarkers detected in an individual are facilitated by the subject devices, systems and methods. Furthermore, the early detection of biomarkers associated with diseases is facilitated by the high sensitivity of the subject devices and systems, as described above. Due to the capability of detecting multiple biomarkers in a single device, combined with sensitivity, scalability, and ease of use, the presently disclosed devices, systems and methods find use in portable and point-of-care or near-patient molecular diagnostics.

The subject devices, systems and methods find use in detecting biomarkers for a disease or disease state. In certain instances, the subject devices, systems and methods find use in detecting biomarkers for the characterization of cell signaling pathways and intracellular communication for drug discovery and vaccine development. For example, the subject devices, systems and methods may be used to detect and/or quantify the amount and/or distribution of biomarkers in diseased, healthy or benign samples. In certain embodiments, the subject devices, systems and methods find use in detecting biomarkers for an infectious disease or disease state. In some cases, the biomarkers can be molecular biomarkers, such as but not limited to proteins, nucleic acids, carbohydrates, small molecules, and the like.

The subject devices, systems and methods find use in diagnostic assays, such as, but not limited to, the following: detecting and/or quantifying biomarkers or the distribution of biomarkers, as described above; screening assays, where samples are tested at regular intervals for asymptomatic subjects; prognostic assays, where the presence and/or quantity or distribution of a biomarker is used to predict a likely disease course; stratification assays, where a subject's response to different drug treatments can be predicted; efficacy assays, where the efficacy of a drug treatment is monitored; and the like. For example, one or more biomarkers may be detected and monitored over an extended period of time, such as over several days, several weeks or several years. Changes in the presence and/or quantity and/or distribution of the one or more biomarkers may be monitored over an extended period of time.

The subject devices, systems and methods also find use in validation assays. For example, validation assays may be used to validate or confirm that a potential disease biomarker is a reliable indicator of the presence or absence of a disease across a variety of individuals. The short assay times for the subject devices, systems and methods may facilitate an increase in the throughput for screening a plurality of samples in a minimum amount of time. For example, the subject devices, systems and methods find use in probed IEF separation medium for affinity reagent screening. High-throughput microfluidic devices that include a separation medium as described herein may be used to select biomarker isoform-specific affinity reagents, such as specific monoclonal antibodies. Such reagents may be used in ELISA assays for disease-specific biomarker isoforms present in clinical proteinaceous samples. In some cases, reagents may be screened in serial or for their multiplexed (parallel) capability for highly specific binding.

The subject devices, systems and methods also find use in a variety of different applications where separation of one or more constituents (e.g., analytes) in a sample is desired. The constituents in the sample may be separated based on a variety of different separation techniques, such as, but not limited to, electrochromotography, electrophoretic immunoassays, equilibrium separations (including isoelectric and temperature gradient focusing), micellar electrokinetic chromatography, chromatography variants, native electrophoresis, and separation by protein mass under denaturing conditions (e.g., SDS-PAGE). Any of the separation techniques may be coupled to subsequent analyte probing by, for example, antibodies (or variants), lectins, substrates, ligands, lipids, coated particles or dyes. For example, separation based on protein sizing with subsequent antibody probing provides an integrated microfluidic Western blotting device.

In some instances, the subject devices, systems and methods can be used without requiring a laboratory setting for implementation. In comparison to the equivalent analytic research laboratory equipment, the subject devices and systems provide comparable analytic sensitivity in a portable, hand-held system. In some cases, the mass and operating cost are less than the typical stationary laboratory equipment. The subject systems and devices may be integrated into a single apparatus, such that all the steps of the assay, including separation, transfer, labeling and detecting of an analyte of interest, may be performed by a single apparatus. For example, in some instances, there are no separate apparatuses for separation, transfer, labeling and detecting of an analyte of interest. In addition, the subject systems and devices can be utilized in a home setting for over-the-counter home testing by a person without medical training to detect one or more analytes in samples. The subject systems and devices may also be utilized in a clinical setting, e.g., at the bedside, for rapid diagnosis or in a setting where stationary research laboratory equipment is not provided due to cost or other reasons.

### Kits

Aspects of embodiments of the present disclosure further include kits configured for use in the methods described herein. In some instances, the kits include a device as described herein, such as a device that includes a polymeric separation medium. In certain embodiments, the kit may include a packaging configured to contain the device. The packaging may be a sealed packaging, such as a sterile sealed packaging. By “sterile” is meant that there are substantially no microbes (such as fungi, bacteria, viruses, spore forms, etc.). In some instances, the packaging may be configured to be sealed, e.g., a water vapor-resistant packaging, optionally under an air-tight and/or vacuum seal.

Aspects of the present disclosure additionally include kits that further include a buffer. For instance, the kit may include a buffer, such as an electrophoresis buffer, a lysis buffer, an electrophoresis/lysis buffer, a sample buffer, a wash buffer, and the like.

In certain cases, the buffer is an electrophoresis buffer, such as, but not limited to, a Tris buffer, a Tris-glycine, and the like. In some instances, the buffer includes a detergent (such as sodium dodecyl sulfate, SDS).

The kits may further include additional reagents, such as but not limited to, release reagents, denaturing reagents, refolding reagents, detergents, detectable labels (e.g., fluorescent labels, colorimetric labels, chemiluminescent labels, multicolor reagents, enzyme-linked reagents, detection reagents (e.g., avidin-streptavidin associated detection reagents), e.g., in the form of at least one if not more analyte detection reagents (such as first and second analyte detection reagents), calibration standards, radiolabels, gold particles, magnetic labels, etc.), and the like.

In certain embodiments, the kit may include an analyte detection reagent, such as a detectable label, as described herein. The detectable label may be associated with a member of a specific binding pair. Suitable specific binding pairs include, but are not limited to: a member of a receptor/ligand pair; a ligand-binding portion of a receptor; a member of an antibody/antigen pair; an antigen-binding fragment of an antibody; a hapten; a member of a lectin/carbohydrate pair; a member of an enzyme/substrate pair; biotin/avidin; biotin/streptavidin; digoxin/antidigoxin; a member of a DNA or RNA aptamer binding pair; a member of a peptide aptamer binding pair; and the like. In certain embodiments, the member of the specific binding pair includes an antibody. The antibody may specifically bind to an analyte of interest in the separated sample bound to the separation medium. For example, the detectable label may include a labeled antibody (e.g., a fluorescently labeled antibody) that specifically binds to the analyte of interest.

In addition to the above components, the subject kits may further include instructions for practicing the subject methods. These instructions may be present in the subject kits in a variety of forms, one or more of which may be present in the kit. One form in which these instructions may be present is as printed information on a suitable medium, e.g., a piece or pieces of paper on which the information is printed, in the packaging of the kit, in a package insert, etc. Another type of instructions can be a computer readable medium, e.g., CD, DVD, Blu-Ray, computer-readable memory (flash memory), etc., on which the information has been recorded or stored. Yet another type of instructions that may be present is a website address which may be used via the Internet to access the information at a removed site. Any convenient instructions may be present in the kits.

As can be appreciated from the disclosure provided above, embodiments of the present invention have a wide variety of applications. Accordingly, the examples presented herein are offered for illustration purposes and are not intended to be construed as a limitation on the invention in any way. Those of ordinary skill in the art will readily recognize a variety of noncritical parameters that could be changed or modified to yield essentially similar results. Thus, the following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by mass, molecular mass is mass average molecular mass, temperature is in degrees Celsius, and pressure is at or near atmospheric.

### EXAMPLES

### Example 1

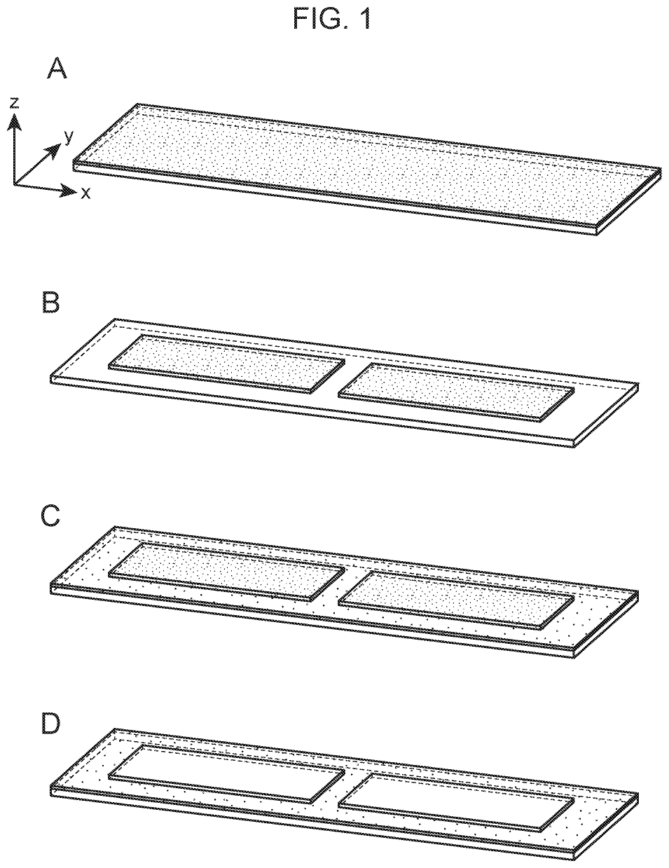

FIG. 1, panels A to D, show a schematic flow diagram illustrating a partial glass silanization process for released gel fabrication, according to embodiments of the present disclosure. As shown in FIG. 1, panel A, a glass slide was covered, for example with Kapton tape. As shown in FIG. 1, panel B, the cover (Kapton tape) was laser cut and the excess portions of the cover were removed, exposing the underlying glass slide where the excess portions of the cover were removed. As shown in FIG. 1, panel C, the exposed portions of the glass slide were silanized. The portions of the glass slide masked by the cover (Kapton tape) were protected from the silanization process. As shown in FIG. 1, panel D, the remaining portions of the cover (Kapton tape) were removed to reveal a partially silanized surface on the glass slide. The partially silanized glass slide was subsequently used in a fabrication process for producing the micropatterned gels used in the tissue projection electrophoresis device, as described in Example 2 below.

### Example 2

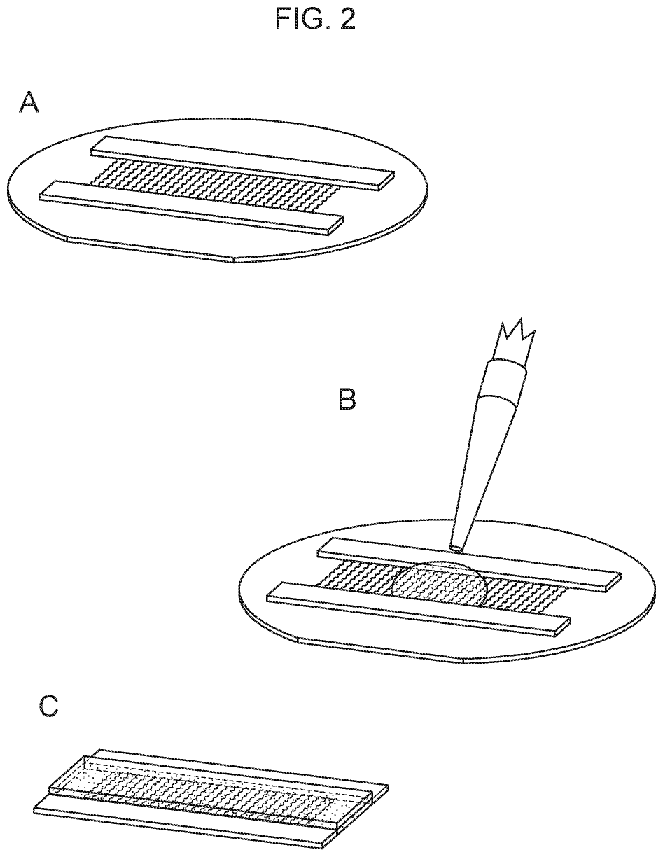

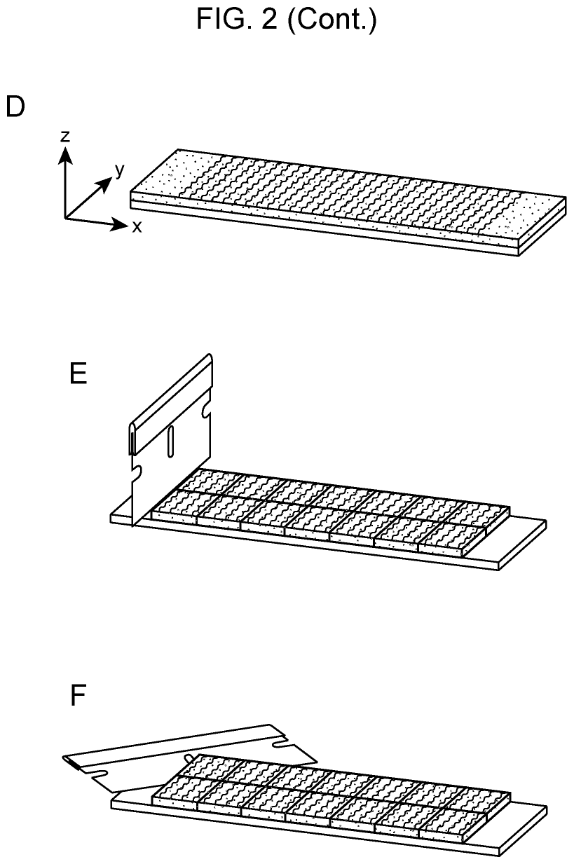

FIG. 2, panels A to F, show a schematic flow diagram illustrating a fabrication process for released, micropatterned gels for Z-directional electrophoresis in a tissue projection device, according to embodiments of the present disclosure. Thick gels were chemically polymerized by molding to a microfabricated SU-8 photoresist on a glass mold, and demolded using the partially-silanized glass slide described above. The partially-silanized glass slide facilitated gel release from the glass after demolding.

As shown in FIG. 2, panel A, spacers (e.g., 1 mm spacers) were affixed to a silicon wafer with SU-8 micropost structures on the surface of the silicon wafer. As shown in FIG. 2, panel B, acrylamide gel precursor solution was added to the micropost structures. As shown in FIG. 2, panel C, the partially-silanized glass slide described above was placed on the spacers. Bubbles were removed from the gel precursor solution, and chemical polymerization of the polyacrylamide gel solution occurred to produce the polyacrylamide gel. As shown in FIG. 2, panel D, the glass slide was removed from the wafer, thus demolding the polyacrylamide gel from the wafer. As shown in FIG. 2, panel E, excess gel was trimmed (for example, using a razor blade) and the gel was diced into freestanding gel pieces. As shown in FIG. 2, panel F, the gels were released from the glass slide, for example by sliding a razor blade between the gels and the glass slide.

### Example 3

FIG. 3 shows a schematic illustration of a tissue projection electrophoresis device, according to embodiments of the present disclosure. As shown in FIG. 3, the cathode (1) and anode (6) provide the electric field for the separation. A gel support (2) for the tissue sample (3) aids in maneuvering the tissue sample (3). The lysis/separation gel (4) provides the lysis buffer to the tissue sample and then serves as the separation matrix, as the electric field drives protein migration through the sieving matrix of the gel (4). A filter paper hydration chamber (5) was soaked in lysis buffer and prevented drying of the small tissue and gel samples during lysis/electrophoresis.

### Example 4

FIG. 4 shows a schematic illustration of an electrophoresis device for analysis of purified proteins, according to embodiments of the present disclosure. As shown in FIG. 4, the cathode (1) and anode (6) provide the electric field for the separation. A shield gel (2) creates a defined volume of purified protein (3) to inject. The separation gel (4) serves as the separation matrix, as the electric field drives protein migration through the sieving matrix of the gel. The separation gel is micropatterned to partition proteins into microwells (via thermodynamic partitioning) for analysis of protein spots in the gel. A filter paper hydration chamber (5) is soaked in lysis buffer and prevents drying of the gel samples during preparation and electrophoresis.

### Example 5

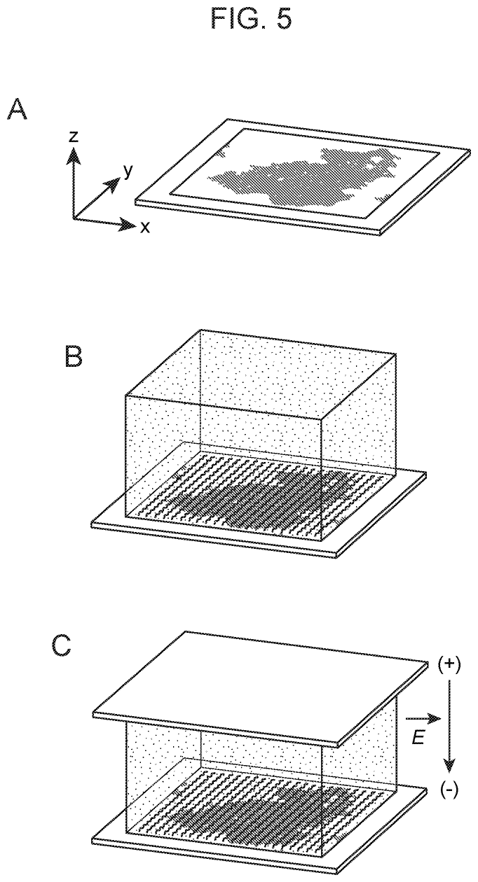

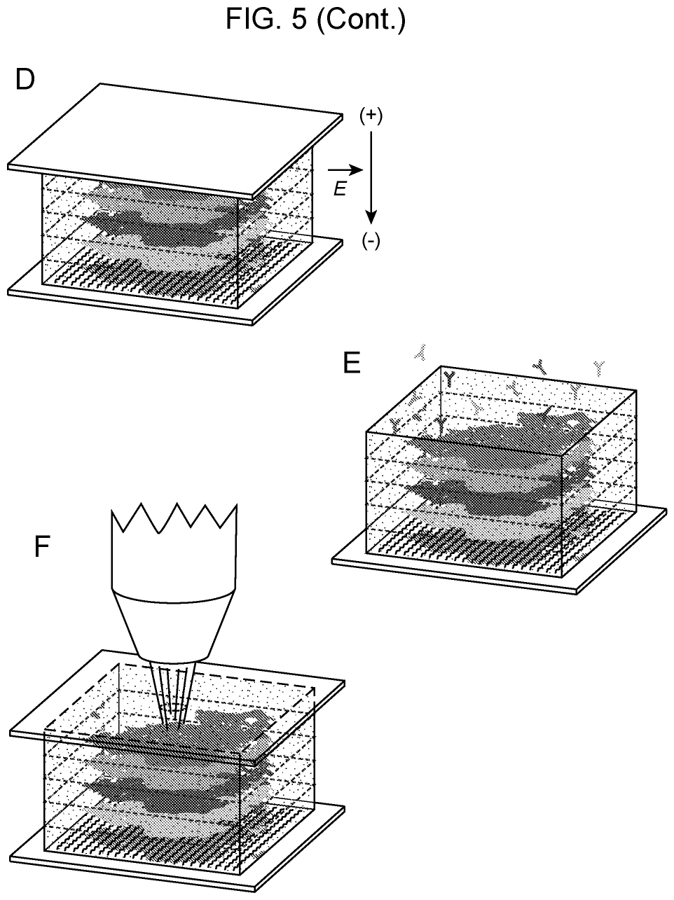

FIG. 5, panels A to F, show a schematic flow diagram illustrating the steps of a tissue projection electrophoresis separation method, according to embodiments of the present disclosure.

As shown in FIG. 5, panel A, a tissue sample (e.g., a tissue slice contained in a gel support) was placed on the bottom electrode. As shown in FIG. 5, panel B, a lysis/separation gel was placed on top of the tissue sample to lyse cells in the tissue. As shown in FIG. 5, panel C, a top electrode was placed on the lysis/separation gel and an electric field was applied. As shown in FIG. 5, panel D, proteins from the tissue sample electrophoretically migrated through the separation gel, separating by size. The electrode setup was then disassembled and the separation gel was exposed to UV light to immobilize the separated proteins to the gel matrix. As shown in FIG. 5, panel E, the separation gel was probed with labelled antibodies after protein immobilization. As shown in FIG. 5, panel F, the gel was imaged using optical sectioning microscopy to obtain the protein projection fingerprint (i.e., map) of the tissue.

### Example 6

The subject device uses Z-directional electrophoresis to project a protein separation in the z-direction while preserving x-y resolution with spatial context information. In order to facilitate z-directional electrophoresis, a released freestanding (support free) gel is used to permit current transport through the gel layers during electrophoresis. Micropatterned gels with microwell features are used to improve x-y resolution by providing lysis micro-volumes to the tissue sample, reducing diffusion in the x-y tissue plane. A released gel fabrication process uses partially-silanized glass slides (fabricated using a Kapton tape mold, in a process shown in FIG. 1, panels A to D) to provide sufficient gel adhesion to release the gel from a photolithographically-patterned mold while still facilitating subsequent release of the gel from the glass. The gel fabrication process is depicted in FIG. 2, panels A to F. The separation gel contains a benzophenone moiety to facilitate photo-immobilization of proteins to the hydrogel matrix.

The device setup for tissue projection electrophoresis is depicted in FIG. 3, while a variant for system characterization using purified protein solutions is shown in FIG. 4. Both setups consist of a stack of free-standing (supportless) gel and sample layers between electrodes. A 1 mm thick hydration chamber that includes of buffer-soaked filter paper surrounds the gels and maintains hydration during electrophoresis in order to reduce edge effects. The parallel plate electrodes are used to provide a consistent electric field for protein migration through the gels. The micropatterned separation gel is used to provide lysis buffer and as the separation medium during electrophoresis, and a flat support gel supports the tissue sample and is used to create a defined purified protein solution volume for characterization experiments. The full process flow schematic is presented in FIG. 5, panels A to F. Briefly, the tissue slice (either in or on a gel support) is placed on the bottom electrode, and the micropatterned lysis/separation gel (soaked in bifunctional lysis/electrophoresis buffer) is brought into contact with the tissue slice to begin lysis. After lysis is complete, an electric field is applied to the gel stack to separate the proteins. After electrophoresis is completed, UV light is applied to immobilize the separated analytes by photocapture of the migrated proteins to the gel matrix. The separation gel is then probed with labelled antibodies and imaged using optical sectioning microscopy to obtain 3D data encompassing both the spatial context information and the electrophoretic protein separation. Instead of using optical sectioning microscopy, the gel could also be histologically sectioned or read out by other techniques yielding 3D maps of the protein distribution in the gel (e.g., lenseless imaging).

The subject devices, systems and methods can also provide for selective lysis of different cell types, different cellular compartments, protein complexes, or cell/matrix proteins via differential detergent fractionation multiplexing. In some instances, the tissue slice can be embedded in a thin, biocompatible hydrogel such as alginate, NiPAAM, or PEGDA. This hydrogel would serve to retain unsolubilized cellular and matrix components between the stages of fractionation. This kind of fractionation would involve conducting consecutive, separate separations using different lysis buffer formulations in distinct separation gels and could facilitate (a) separating proteins by localization within the cell; (b) separating proteins by cell type (as different cell types can require different lysis conditions for effective protein solubilization); (c) optimizing lysis conditions to either retain or dissociate protein complexes, and (d) separating matrix proteins after cellular proteins. Separating matrix proteins may be of interest as post-translational modifications of the extra-cellular matrix (e.g., by protease cleavage and enzyme interaction) have been identified as disease-specific biomarkers, and information regarding these kinds of modifications would provide additional and complementary insight into disease state and cellular behavior and protein expression. This type of information would facilitate a determination of the complex, and dynamic interactions between different cell types and the extra-cellular matrix within tissue.

The subject devices, systems and methods can also be used for other types of protein separation in the Z direction while maintaining spatial context. For example, separating proteins by charge (isoelectric point) via isoelectric focusing can facilitate detecting difficult-to-measure proteoforms (e.g., those with similar molecular weights, such as changes in phosphorylation state) in a tissue sample.

Although the foregoing embodiments have been described in some detail by way of illustration and example for purposes of clarity of understanding, it is readily apparent to those of ordinary skill in the art in light of the teachings of the present disclosure that certain changes and modifications may be made thereto without departing from the spirit or scope of the appended claims. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

Accordingly, the preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of present invention is embodied by the appended claims.

That which is claimed is:

1. A method of detecting a distribution of analytes in a tissue sample, the method comprising:
(a) applying an electric field to a tissue sample on a polymeric separation medium in a manner sufficient to move one or more analytes from the tissue sample into the polymeric separation medium to separate the analytes in the polymeric separation medium;
(b) exposing the polymeric separation medium to an applied stimulus to immobilize the separated analytes in the polymeric separation medium; and
(c) detecting the analytes to produce a map of the distribution of the analytes in the tissue sample.

2. The method of claim 1, wherein the map is a three-dimensional distribution of the analytes in the tissue sample.

3. The method of claim 1, wherein the polymeric separation medium comprises a buffer sufficient to differentially lyse different cell types in the tissue sample.

4. The method of claim 1, wherein the polymeric separation medium comprises a buffer sufficient to differentially lyse a subcellular compartment of one or more cells in the tissue sample.

5. The method of claim 1, wherein the polymeric separation medium comprises a buffer sufficient to either retain or dissociate protein complexes in the tissue sample.

6. The method of claim 1, wherein the polymeric separation medium comprises a buffer sufficient for separation of cellular proteins from matrix proteins in the tissue sample.

7. The method of claim 1, wherein the detecting comprises contacting the separated analytes with an analyte detection reagent.

8. The method of claim 7, wherein the detecting further comprises imaging the polymeric separation medium to produce an image of the separated analytes in the polymeric separation medium.

9. The method of claim 1, further comprising assembling a device for performing the method.

10. The method of claim 9, wherein the assembling comprises:
(1) disposing the tissue sample on a first electrode;
(2) positioning the tissue sample in fluid communication with a first surface of the polymeric separation medium; and
(3) disposing a second electrode on an opposing second surface of the polymeric separation medium.

11. The method of claim 10, wherein the tissue sample is disposed on a surface of a support medium, or wherein the tissue sample is contained in a support medium.

12. A device for detecting a distribution of analytes in a tissue sample, the device comprising:
a tissue sample comprising one or more analytes; and
a polymeric separation medium comprising a buffer and functional groups that covalently bond the analytes in the tissue sample to the polymeric separation medium upon application of an applied stimulus.

13. The device of claim 12, wherein one surface of the tissue sample is in contact with a surface of the polymeric separation medium and an opposing surface of the tissue sample is in contact with a support medium.

14. The device of claim 12, wherein the tissue sample is contained in a support medium, and a surface of the support medium is in contact with a surface of the polymeric separation medium.

15. The device of claim 12, wherein the polymeric separation medium comprises an array of microwells in a surface of the polymeric separation medium facing the tissue sample.

16. The device of claim 12, wherein the functional groups comprise light-activated benzophenone functional groups.

17. The device of claim 12, wherein the polymeric separation medium comprises one or more separated analytes from the tissue sample, and the separated analytes in the polymeric separation medium are correlated to a distribution of the analytes in the tissue sample.

18. The device of claim 12, further comprising a chamber containing the tissue sample and the polymeric separation medium.

19. A kit for performing the method of claim 1, wherein the kit comprises:
a device comprising the polymeric separation medium; and
a packaging containing the device.

20. The kit of claim 19, further comprising one or more buffers.
